(19) United States
(12) Patent Application Publication
(10) Pub. No.: US 2026/0183748 A1
(43) Pub. Date: Jul. 2, 2026

(54) LEWIS ACID BASE PAIRS AS HIGHLY ACTIVE CATALYTIC SITES FOR HYDROGENATION AND DEHYDROGENATION PROCESSES

(71) Applicants: BASF Corporation, Florham Park, NJ (US); The Regents of the University of California, Oakland, CA (US)

(72) Inventors: Vardan Danghyan, Berkeley, CA (US); Nicholas Jaegers, Berkeley, CA (US); Carlos Lizandara Pueyo, Ludwigshafen am Rhein (DE); Craig Jon Cain-Borgman, Iselin, NJ (US); Joseph C. Dellamorte, Wyandotte, MI (US); Arunabha Kundu, Beachwood, OH (US); Junnan Shangguan, Berkeley, CA (US); Enrique Iglesia, Berkeley, CA (US)

(21) Appl. No.: 18/835,206

(22) PCT Filed: Feb. 20, 2024
(86) PCT No.: PCT/US2024/016500

§ 371 Date: Aug. 1, 2024

Related U.S. Application Data
(60) Provisional application No. 63/447,138, filed on Feb. 21, 2023.

(51) Int. Cl. B01J 21/06; B01J 21/08; B01J 23/10; B01J 23/75; B01J 35/61; B01J 37/02; B01J 37/03; B01J 37/04; B01J 37/06; B01J 38/52; C07C 5/03; C07C 5/333; C07C 9/08; C07C 9/12; C07C 11/06; C07C 11/09

(52) U.S. Cl. CPC B01J 21/066; B01J 21/08; B01J 23/10; B01J 23/75; B01J 35/613; B01J 37/0201; B01J 37/031; B01J 37/04; B01J 37/06; B01J 38/52; C07C 5/03; C07C 5/3332; C07C 9/08; C07C 9/12; C07C 11/06; C07C 11/09; C07C 2521/06; C07C 2523/10; C07C 2523/75

(57) ABSTRACT

Methods of treating a reactant stream including a catalyst composition comprising a metal oxide (MOₓ) catalyst having a surface with an M-O site of the Lewis type and of balanced acid-base strength and a high surface area and a trap are disclosed. The trap may remove an impurity from the reactant stream. The MOₓ catalyst includes a BET surface area of at least about 50 m²/gram.

### CROSS REFERENCE TO RELATION APPLICATION(S)

The present application claims priority to U.S. Provisional Patent Application No. 63/447,138 filed on Feb. 21, 2023, the entire contents of which is incorporated in its entirety.

### FIELD OF THE INVENTION

Disclosed herein are methods of treating a reactant stream using a catalyst composition including a metal oxide (MOₓ) catalyst and a trap for removal of impurities from the reactant stream. In particular, the method includes treating the reactant stream with the trap, wherein the trap may remove an impurity, and the MOₓ facilitates chemical reactions. The impurity may include oxygen (O₂), water (H₂O), hydrogen sulfide (H₂S), carbon dioxide (CO₂), carbon monoxide (CO), nitrogen (N₂), a sulfur containing compound, methanol or other alcohols, a ketone, an inorganic nitrogen compound, an organic nitrogen compound, an oxygenate, or a combination thereof. The MOₓ catalyst may have a surface with a M-O site of the Lewis type and of balanced acid-base strength. The MOₓ catalyst may have a BET surface area of at least about 75 m²/gram.

### BACKGROUND

In the chemical industry, propene is an important intermediate for the production of a variety of compounds. For example, propene may be used in the production of propylene oxide, acrylonitrile, cumene, acrylic acid, C₃ and larger alcohols, and polypropylene. Historically, propene has been available as a co-product from steam crackers and off-gas product from fluid cracking catalytic units in oil refineries. These technologies have been found to be insufficient to meet the demands of propene, leading to the development of new technologies, such as dehydrogenation.

Thus, dehydrogenation technologies and catalysts have been extensively developed and commercialized in recent years. Among them, the propene dehydrogenation (PDH) has been one of the most common alternate technologies because of its standalone suitability. Non-limiting representative PDH technologies include those employing platinum group metal (PGM) catalysts (e.g., Linde-BASF, Oleflex, STAR, and FCDh (DOW) processes), or Cr-containing catalysts (e.g., Catofin and FDB-4 processes). In recent years, alternate materials for the catalysis have been explored to address the toxicity and environmental hostility of Cr-based systems and/or could reduce or replace the high costs associated to the PGM catalysts.

Metal oxides, such as ZrO₂, are promising alternatives for use in a catalyst composition because of the presence of Lewis acid-base (LAB) pairs. A major limitation in the use of metal oxides for PDH is their fast deactivation. During the dehydrogenation of hydrocarbons such as propane, the acid-base pairs of metal oxide dehydrogenation catalysts (including ZrO₂) may be deactivated due to (i) titration by H₂O and/or CO₂, which directly derive from the gas feed streams or are formed indirectly via reactions of O₂ or oxygenate impurities from gas streams with propane and/or H₂; and/or (ii) coke deposition resulting from the adsorption of paraffin-derived molecules on M-O sites. In addition, due to the reducibility of some metal oxides, metal oxide dehydrogenation catalysts such as, e.g., TiO₂ and MoOₓ can be reduced to a lower oxidation state or even the metallic state over time, potentially resulting in deactivation or activation.

Efforts have been made to develop regeneration/reactivation processes for these catalysts. For example, metal oxide catalysts with surfaces rendered at least partially inactive by bound CO₂ and/or H₂O may be activated and/or reactivated using high temperature thermal regeneration processes. However, loss of activity due to sintering and annealing during the thermal regeneration process is not reversible.

Accordingly, methods have been developed to clean the surface of the MOₓ catalyst including acid-base sites, where the cleaning of the MOₓ catalyst creates active sites on the surface without inducing the significant sintering or annealing noted in high temperature thermal regeneration processes. It has been found that these surfaces may be re-titrated, such that the surface including the acid-base site adsorbs and/or absorbs impurities and limits the reactivity time of such catalysts.

That is, the chemical cleaning of these surfaces creates active surfaces, but such surfaces can be titrated again by trace hydroxyls (from H₂O) or carbonated/bicarbonates (from CO₂) impurities in reactant streams or by molecules such as O₂ that can form such titrants during paraffin dehydrogenation. The prevention of the titration of active sites because of impurities present in the inlet gas is a feasibility issue to be addressed for practical processes involving LAB pair sites. The chemical treatment with dimethyl ether (DME) has led to unprecedented and practical turnover rates for ZrO₂ on materials not previously considered practical as PDH catalysts. However, the preservation of such reactivity with time requires the removal of impurities from the reaction system during operation of paraffin dehydrogenation and related processes.

Thus, there is a need for a method of pretreating and/or treating the reactant stream to remove impurities and increase the lifetime and performance of such catalysts for the dehydrogenation/hydrogenation reactions.

### SUMMARY

Disclosed herein is a method of treating a reactant stream using a catalyst composition including a metal oxide (MOₓ) catalyst and a trap, wherein the reactant stream may include a hydrocarbon. The trap may remove an impurity from the reactant stream. The impurity may include oxygen (O₂), water (H₂O), hydrogen sulfide (H₂S), carbon dioxide (CO₂), carbon monoxide (CO), nitrogen (N₂), a sulfur containing compound, methanol or other organic alcohols, a ketone, an inorganic nitrogen compound, an organic nitrogen compound, an oxygenate, or a combination thereof. The MOₓ catalyst may have a surface with a M-O site of the Lewis type and of balanced acid-base strength. The MOₓ catalyst may have a BET surface area of at least about 75 m²/gram.

In some embodiments, the impurity may be removed from a reactant stream before the stream contacts the catalyst composition during a hydrogenation or dehydrogenation process.

In some embodiments, a surface of the MOₓ catalyst may stabilize anionic and/or cationic moieties that form at transition states for heterolytic processes that form and cleave C—H bonds.

In some embodiments, a metal (M) of the MOₓ catalyst does not undergo reduction to a lower oxidation state in a reductive environment typical of hydrogenation-dehydrogenation catalysis.

In some embodiments, the method may operate at an impurity content of less than about 2 ppm.

In some embodiments, the method may operate at an impurity content of at most 100 ppm, at most 50 ppm, or at most 20 ppm.

In some embodiments, the reactant stream may have an oxygen content of about 5 ppm, about 1 ppm, or about 0.2 ppm.

In some embodiments, the surface area of the MOₓ catalyst may be proportional to density of active sites of the MOₓ catalyst.

In some embodiments, the MOₓ catalyst comprises a crystalline active component, wherein when the surface area of the crystalline active component decreases, then a density of active sites of the MOₓ catalyst decreases.

In some embodiments, the MOₓ catalyst may be substantially free of hydroxyl groups.

In some embodiments, the treated MOₓ catalyst does not contain hydroxyl groups.

In some embodiments, the trap may be an oxygen trap, water trap, carbon dioxide trap, an impurity trap, or a combination thereof.

In some embodiments, the surface with the M-O site of the Lewis type and of the balanced acid-base strength may be maintained during a dehydrogenation or hydrogenation process by avoiding titration with the impurity.

In some embodiments, the treating of the reactant stream may be performed at a temperature of about 500-900 K.

In some embodiments, the treating of the reactant stream may be performed at a temperature up to 900 K.

In some embodiments, the MOₓ catalyst may include a metal oxide having metal centers in cationic form.

In some embodiments, the MOₓ catalyst may include a metal oxide in which the cations are not reducible to a zero-valent state.

In some embodiments, the MOₓ catalyst may include a metal selected from the group consisting of zirconium (Zr), cobalt (Co), gallium (Ga), zinc (Zn), cerium (Ce), yttrium (Y), hafnium (Hf), and titanium (Ti).

In some embodiments, the MOₓ catalyst may include one or more of Mg, Ca, Sr, Ba and La on a zirconia support.

In some embodiments, the MOₓ catalyst may include ZrO₂-silica, Zr—Al, Zr—Ti, or a combination thereof.

In some embodiments, the MOₓ catalyst may include ZrO₂, tetragonal ZrO₂ (t-ZrO₂), monoclinic ZrO₂ (m-ZrO₂), Y-stabilized ZrO₂, Ce-stabilized ZrO₂ or Y₂O₃.

In some embodiments, the method may include cleaning the MOₓ catalyst with a surface cleaning reagent.

In some embodiments, the surface cleaning reagent may include dimethyl ether, propylene, ethylene, methanol, tert-butyl alcohol, methyl tert-butyl ether, di-tert-butyl ether, anisole, dimethyl carbonate, or combinations thereof. In another embodiment, the surface cleaning reagent may include dimethyl ether. In yet another embodiment, the surface cleaning reagent may include methanol. In some embodiments, the surface cleaning reagent may include an alkene. In another embodiment, the surface cleaning reagent may include propylene.

In some embodiments, the method may further include cleaning the MOₓ catalyst during the dehydrogenation or hydrogenation rection by balancing water deposition and water removal by the alkene products at a temperature of about 800-900 K.

In some embodiments, the cleaning may be performed at a temperature between about 323 K and 900 K.

In another embodiment of the present disclosure, a method of catalyzing a reaction using a catalyst composition as described herein is also provided. In an embodiment, a method of catalyzing a reaction using a catalyst composition including a MOₓ catalyst and a trap, including activating and/or reactivating the catalyst composition using the method of treating as described herein. The reaction may be selected from the group consisting of alkane dehydrogenation, alkene hydrogenation, olefin-paraffin alkylation, reactions of CO/H₂ mixtures without O-rejection as H₂O or CO₂, C—C bond formation via alkene oligomerization or metathesis, dehydrocyclization (alkanes/alkenes to arenes), dehydrocyclodimerization (alkanes/alkenes to arenes with a larger number of C-atoms), transfer hydrogenations, hydroformylation/carbonylation, aromatization, dearomatization, reforming, isomerization, and bifunctional reactions in which one of the aforementioned functions can be optionally combined with a Brønsted acid function.

In some embodiments, the treating of the catalyst composition may be performed simultaneously with the catalyzing reaction.

In some embodiments, the reaction may be alkane dehydrogenation. In another embodiment, the reaction may be alkene hydrogenation.

In some embodiments, the MOₓ catalyst may include a metal oxide having metal centers in cationic form.

In some embodiments, the MOₓ catalyst may include a metal oxide that is not reducible to zero-valent state.

In some embodiments, the MOₓ catalyst may include a metal selected from the group consisting of Zr, Co, Ga, Zn, Ce, Y, Hf, and Ti.

In some embodiments, the catalyst composition may improve product yield at least 2-fold compared with a comparable reaction using a catalyst composition that does not include a trap.

In some embodiments, the method may further include cycling between dehydrogenating the light alkane gas or hydrogenating the light alkene gas with the catalyst composition and reactivating the catalyst composition.

In another embodiment, a catalyst composition is provided. In an embodiment, the catalyst composition may include a MOₓ catalyst having a surface with an M-O site of the Lewis type and of balanced acid-base strength and a BET surface area of at least about 50 m²/gram, wherein the catalyst composition may be free of at least one of chromium or a precious metal.

In some embodiments, the MOₓ catalyst may include a metal oxide having metal centers in cationic form.

In some embodiments, the MOₓ catalyst may include a metal oxide that is not reducible to zero-valent state.

In some embodiments, the MOₓ catalyst may include a metal selected from the group consisting of Zr, Co, Ga, Zn, Ce, Y, and Ti.

In some embodiments of the catalyst composition, the MOₓ catalyst may include ZrO₂. In some embodiments, the catalyst composition may include at least about 25 wt % ZrO₂ based on total weight of the catalyst composition.

In some embodiments, the catalyst composition may further include a rare earth metal comprising at least one lanthanide metal, an oxide thereof, or combinations thereof. In another embodiment, the catalyst composition may include a rare earth metal comprising at least one of Y, erbium (Er), Ce, dysprosium (Dy), gadolinium (Gd), lanthanum (La), neodymium (Nd), samarium (Sm), ytterbium (Yb), oxides thereof or mixtures thereof.

In some embodiments, the catalyst composition may include about 0.5 wt % to about 50 wt % of the rare earth metal.

In some embodiments, the catalyst composition may be cleaned and may include more surface-active sites than before cleaning.

### BRIEF DESCRIPTION OF THE DRAWINGS

FIG. 1 depicts the ZrO₂ surface area (circles, left axis) and particle size (triangles, right axis) after treatment at various temperatures;

FIG. 2 depicts the effect of ZrO₂ particle size on propane dehydrogenation rates (15 kPa C₃H₈, 5 kPa H₂, 723K) per catalyst mass after thermal treatment and chemical cleaning;

FIG. 3 depicts the effect of the ZrO₂ particle size on propane dehydrogenation rates (15 kPa C₃H₈, 5 kPa H₂, 723K) per mass for DME treated ZrO₂ samples;

FIG. 4 depicts the active site titrations by H₂O pulse injections during dehydrogenation reaction (15 kPa C₃H₈, 5 kPa H₂, 723 K) for ZrO₂ treated thermally at different temperatures followed by DME treatment;

FIG. 5 depicts the effect of the ZrO₂ particle size on the number of active sites per nm²;

FIG. 6, the graph shows a linear decrease in rates for pure monoclinic and tetragonal ZrO₂ as a function of the amount of water injected with the x-axis intersect defining the number of water molecules required to suppress reactivity

FIG. 7 depicts isobutane dehydrogenation rates (per mass) on ZrO₂ treated at different temperatures in He for 30 minutes followed by subsequent DME treatments;

FIG. 8 depicts isobutane dehydrogenation rates (areal) on ZrO₂ treated at different treatment temperatures in He for 30 minutes with subsequent DME treatments;

FIG. 9 illustrates the XRD patterns of synthesized monoclinic ZrO₂ samples according to Example 1 after treatment at 573 K and 723 K, and for tetragonal Y—ZrO₂ samples according to Example 2 at 573 K;

FIG. 10 depicts alkane dehydrogenation rate constants for C₂-C₇ alkanes after He (lower) and DME (upper) treatments at 723 K on Example 1;

FIG. 11 depicts active site titration by O₂ and H₂O during propane dehydrogenation catalysis (15 kPa C₃H₈, 5 kPa H₂, 723 K) ZrO₂ of Example 1;

FIG. 12 depicts propane dehydrogenation areal rate in μmol m⁻² h⁻¹ vs. Time on Stream in ks and the mean-life of active sites at 723 K for DME treated m-ZrO₂ of Example 1 with reactant stream bypassing or flowing through the oxygen trap;

FIG. 13 depicts propane dehydrogenation areal rate in in μmol m⁻² h⁻¹ vs. Time on Stream in ks after He (lower) and DME (upper) treatments at 723 K on ZrO₂ of Example 1 with reactant stream passing through a trap;

FIG. 14 depicts propane dehydrogenation areal rate in μmol m⁻² h⁻¹ vs. Time on Stream in ks after He (lower) and DME (upper) treatments at 723 K on Y—ZrO₂ of Example 2 with reactant stream passing through a trap;

FIG. 15 depicts propane dehydrogenation forward rate in mol kg⁻¹ h⁻¹ vs. Time on Stream in ks at 723 K (lower) and 873 K (upper) with an oxygen trap and for a short duration of bypassing the trap at 873 K;

FIG. 16 depicts propane dehydrogenation rate in mol kg⁻¹ h⁻¹ vs. Time on Stream in ks on ZrO₂ upon increasing the temperature from 723 K to 873 K and decreasing it back to 723 K during propane dehydrogenation with the reactant stream passing through the trap at the following reaction conditions: 15 kPa, C₃H₈, 5 kPa H₂;

FIG. 17 depicts the results of Example 18;

FIG. 18 depicts propane dehydrogenation areal rate in μmol m⁻² h⁻¹ after DME, He and H₂ treatments at the following conditions: 13.7 kPa propane, 723 K;

FIG. 19 depicts propane dehydrogenation rates areal rate in μmol m⁻² h⁻¹ as function of propane and hydrogen pressure on the sample of Example 1 at 723 K;

FIG. 20 depicts propene hydrogenation rates areal rate in μmol m⁻² h⁻¹ as a function of propene and H₂ pressure on the sample of Example 1 at 723 K; and

FIG. 21 depicts extracted rate constants in mol Kg⁻¹ bar⁻¹ or⁻² vs. 10³ K/T and activation barriers for propane dehydrogenation (triangle) and propene hydrogenation (circle). Propene hydrogenation rate constants are compared to predicted hydrogenation rate constants using gas phase thermodynamic connections to dehydrogenation rate constants (triangles).

### Definitions

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein.

As used herein, the term “and/or” includes any and all combinations of one or more of the associated listed items. Additionally, as used herein, “or” means “and/or.”

The term “alkyl,” as used herein, refers to a saturated straight-chain (i.e., linear or unbranched) or branched hydrocarbon chain containing carbon atoms (such as, e.g., 1, 2, 3, 4, 5, 6, 7, 8, 9, 10, 11, 12, 13, 14, 15, 16, 17, 18, 19, or 20 carbon atoms). Unless otherwise specified, alkyl groups contain 1-20 carbon atoms. In some embodiments, alkyl groups contain 1-10 carbon atoms (denoted as C₁₋₁₀ alkyl herein). In some embodiments, alkyl groups contain 1-8 carbon atoms (denoted as C₁₋₈ alkyl herein). In some embodiments, alkyl groups contain 1-6 carbon atoms (denoted as C₁₋₆ alkyl herein). In some embodiments, alkyl groups contain 1-4 carbon atoms (denoted as C₁₋₄ alkyl herein). In some embodiments, alkyl groups contain 1-3 carbon atoms (denoted as C₁₋₃ alkyl herein). Nonlimiting examples of “alkyl” groups include methyl, ethyl, propyl, isopropyl, isobutyl, tert-butyl, sec-butyl, and the like.

The term “alkenyl,” as used herein, means a straight-chain (i.e., linear or unbranched) or branched hydrocarbon chain that contains at least one carbon-carbon double bond. Unless otherwise specified, alkenyl groups contain 2-20 (such as, e.g., 2-12, 2-6, or 2-4) carbon atoms. Nonlimiting examples of “alkenyl” groups include vinyl, allyl, butenyl, pentenyl, hexenyl, butadienyl, pentadienyl, hexadienyl, 2-ethylhexenyl, cyclopent-1-en-1-yl, and the like.

The term “alkynyl,” as used herein, means a straight-chain (i.e., linear or unbranched) or branched hydrocarbon chain that contains at least one carbon-carbon triple bond. Unless otherwise specified, alkynyl groups contain 2-20 (such as, e.g., 2-12, 2-6, or 2-4) carbon atoms. Nonlimiting examples of “alkynyl” groups include ethynyl, propynyl, butynyl, pentynyl, hexynyl, and the like.

The term “aryl” refers to monocyclic, bicyclic, and tricyclic ring systems having a total of five to fourteen ring members, wherein at least one ring in the system is aromatic and wherein each ring in the system contains 3 to 7 ring members. The term “aryl,” as used herein, also refers to heteroaryl ring systems as defined herein below.

As used herein, the term “catalyst composition” refers to a composition comprising a material that promotes a chemical reaction.

The term “heteroatom,” as used herein, refers to an oxygen, sulfur, nitrogen, phosphorus, or silicon (including, any oxidized form of nitrogen, sulfur, phosphorus, or silicon; the quaternized form of any basic nitrogen; or a substitutable nitrogen of a heterocyclic ring, e.g., N (as in 3,4-dihydro-2H-pyrrolyl), NH (as in pyrrolidinyl), or NR⁺ (as in N-substituted pyrrolidinyl)) atom.

The term “heteroaryl,” as used herein, refers to a monocyclic, bicyclic, and tricyclic ring system, including fused or bridged ring systems, having a total of five to fourteen ring members, wherein at least one ring in the system is aromatic, at least one ring in the system contains one or more heteroatoms, and wherein each ring in the system contains 3 to 7 ring members. Non-limiting examples of “heteroaryl” groups include azepinyl, acridinyl, benzimidazolyl, benzothiazolyl, benzindolyl, cinnolinyl, dibenzofuranyl, dibenzothiophenyl, furanyl, isothiazolyl, imidazolyl, indazolyl, indolyl, isoindolyl, indolinyl, isoquinolyl, indolizinyl, isoxazolyl, naphthyridinyl, oxadiazolyl, oxazolyl, pyrrolyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl).

As used herein, the term “increase” refers to altering positively, including, but not limited to, altering positively by 1%, altering positively by 5%, altering positively by 10%, altering positively by 25%, altering positively by 30% altering positively by 50%, altering positively by 75%, altering positively by 100%, altering positively by 200%, and the like.

As used herein, the term “decrease” refers to altering negatively, including, but not limited to, altering negatively by 1%, altering negatively by 5%, altering negatively by 10%, altering negatively by 25%, altering negatively by 30%, altering negatively by 50%, altering negatively by 75%, or altering negatively by 100%.

As used herein, the term “pretreating” refers to any process in which a catalyst is contacted with a chemical, combination of chemicals, or a series of chemicals to remove an impurity from the surface of a catalyst. As used herein, the term “cleaning” refers to any process in which a catalyst is contacted with a chemical, combination of chemicals, or a series of chemicals to activate or reactivate the catalyst to a higher activity and/or selectivity state, either before using the catalyst for the intended chemical process or at intervening points in time during use of the catalyst. In some embodiments, pretreating is carried out inside a chemical reactor. In some embodiments, pretreating is carried out outside a chemical reactor. In some embodiments, when used at intervening points during catalyst use, pretreating restores all or a portion of the activity and/or selectivity of the catalyst in protocols that may be denoted to those skilled in the art as catalyst regeneration treatments.

As used herein, the term “impurity free” or “substantially free” refers to stream that comprises less than about 1 wt %, less than about 0.5 wt %, less than about 0.25 wt %, less than about 0.1 wt %, less than about 0.05 wt %, less than about 0.01 wt %, or 0 wt % of a component, such as an impurity.

### DETAILED DESCRIPTION OF THE PRESENT DISCLOSURE

In an embodiment of the present disclosure, a method of treating a reactant stream using a catalyst composition comprising a MOₓ catalyst and a trap. The method may include treating the reactant stream with the trap, wherein the trap may remove an impurity. The impurity may include O₂, H₂O, H₂S, CO₂, CO, N₂, a sulfur containing compound, methanol or other alcohol, a ketone, an inorganic nitrogen compound, an organic nitrogen compound, an oxygenate, or a combination thereof. The MOₓ catalyst may have a surface with a M-O site of the Lewis type and of balanced acid-base strength. The MOₓ catalyst may have a BET surface area of at least about 75 m²/gram. In some embodiments, the BET surface area may be about 50 m²/gram, about 55 m²/gram, about 60 m²/gram, about 65 m²/gram, about 70 m²/gram, about 75 m²/gram, about 80 m²/gram, about 85 m²/gram, about 90 m²/gram, about 95 m²/gram, about 100 m²/gram, about 105 m²/gram, about 110 m²/gram, about 115 m²/gram, about 120 m²/gram, about 125 m²/gram, about 130 m²/gram, about 135 m²/gram, about 140 m²/gram, about 145 m²/gram, about 150 m²/gram, about 155 m²/gram, about 160 m²/gram, about 165 m²/gram, about 170 m²/gram, about 175 m²/gram, about 180 m²/gram, about 185 m²/gram, about 190 m²/gram, about 195 m²/gram, about 200 m²/gram, or greater.

In some embodiments, the impurity may be removed from a reactant stream before it contacts the catalyst composition during a dehydrogenation or hydrogenation process. The impurity may be any species that can form H₂O, NH₃, or H₂S during dehydrogenation reactions. For example, but without being limited to, the impurity may be O₂, CO, N₂, oxygenates, an organonitrogen compound, or an organosulfur compound because these molecules may react with reactants and form titrants.

In some embodiments, a surface of the MOₓ catalyst may stabilize anionic and/or cationic moieties that form at transition states for heterolytic processes that form and cleave C—H bonds.

In some embodiments, the MOₓ catalyst possesses a surface with a M-O site of the Lewis type and of balanced acid-base strength; a surface of the MOₓ catalyst stabilizes anionic and/or cationic moieties that form at transition states for heterolytic processes that form and cleave C—H bonds; and a metal (M) of the MOₓ catalyst does not undergo reduction to a lower oxidation state in a reductive environment typical of hydrogenation-dehydrogenation catalysis.

In some embodiments, M of the MOₓ catalyst does not undergo reduction to a lower oxidation state in a reductive environment typical of hydrogenation-dehydrogenation catalysis.

In some embodiments, the method of the present disclosure may operate at an impurity content of less than about 2 ppm. In some embodiments, the impurity content may be about 0.0001 ppm to about 2 ppm, about 0.001 ppm to about 1.5 ppm, about 0.05 ppm to about 1 ppm, about 0.1 ppm to about 0.9 ppm, about 0.2 ppm to about 0.8 ppm, about 0.3 ppm to about 0.7 ppm, or about 0. 4 ppm to about 0.6 ppm. In other embodiments, the method may operate at an impurity content of less than about 0.5 ppm, about 0.4 ppm, about 0.3 ppm, about 0.2 ppm, about 0.1 ppm, or about 0.05 ppm.

In some embodiments, the method may operate at an impurity content of at most 100 ppm, at most 50 ppm, or at most 20 ppm. In other embodiments, the method may operate at an impurity content of at most about 100 ppm, at most about 90 ppm, at most about 80 ppm, at most about 70 ppm, at most about 60 ppm, at most about 50 ppm, at most about 40 ppm, at most about 30 ppm, or at most about 20 ppm. In some embodiments, the impurity content may be about 0.0001 ppm to about 100 ppm, about 0.001 ppm to about 95 ppm, about 0.01 ppm to about 90 ppm, about 0.1 ppm to about 85 ppm, about 1 ppm to about 80 ppm, about 5 ppm to about 75 ppm, about 10 ppm to about 70 ppm, about 15 ppm to about 65 ppm, about 20 ppm to about 60 ppm, about 25 ppm to about 55 ppm, about 30 ppm to about 50 ppm, or about 35 ppm to about 45 ppm.

In some embodiments, the reactant stream may have an oxygen content of about 5 ppm, about 4.5 ppm, about 4 ppm, about 3.5 ppm, about 3 ppm, about 2.5 ppm, about 2 ppm, about 1.5 ppm, about 1 ppm. In other embodiments, the reactant stream may have an oxygen content of about 0.5 ppm, about 0.45 ppm, about 0.4 ppm, about 0.35 ppm, about 0.3 ppm, about 0.25 ppm, about 0.2 ppm, about 0.15 ppm, or about 0.1 ppm. In yet another embodiment, the reactant stream may have an oxygen content of about 0.1 ppm to about 2 ppm, about 0.15 ppm to about 1.75 ppm, about 0.2 ppm to about 1.5 ppm, about 0.25 ppm to about 1.25 ppm, about 0.3 ppm to about 1 ppm, about 0.35 ppm to about 0.75 ppm, or about 0.4 ppm to about 0.5 ppm.

In some embodiments, the density of active sites may be proportional to the surface area of the MOₓ catalyst. It has been found that to maintain a large number of active sites per catalyst mass in a catalyst composition, sintering of the catalyst composition should be avoided because this causes both loss of surface area and a decrease in the areal density of active sites, the latter because of the annealing of specific LAB pair active sites.

In some embodiments, when the surface area decreases, then a density of active sites of the MOₓ catalyst may decrease.

In some embodiments, the MOₓ catalyst may be substantially free of hydroxyl groups.

In some embodiments, the MOₓ catalyst does not contain hydroxyl groups.

In some embodiments, the reactant stream may be substantially free of oxygen after contacting the catalyst composition including a MOₓ catalyst and trap. In other embodiments, the reactant stream does not contain oxygen after contacting the catalyst composition including a MOₓ catalyst and trap.

In some embodiments, the trap may be an oxygen trap, water trap, carbon dioxide trap, or a combination thereof.

In some embodiments, the surface with the M-O site of the Lewis acid-base pair type and with balanced acid-base strength may be maintained during a dehydrogenation or hydrogenation process by avoiding titration with the impurity.

In some embodiments, the treating of the reactant stream may be performed at a temperature of about 500-900 K. In some embodiments, the treating of the reactant stream may be performed at a temperature of about 500 K to about 900 K, about 550 K to about 850 K, about 600 K to about 800 K, or about 650 K to about 750K.

In some embodiments, the treating of the reactant stream may be performed at a temperature up to 900 K. In some embodiments, the treating may be performed at a temperature of about 500 K, about 550 K, about 600 K, about 650 K, 700 K, about 750 K, about 800 K, about 850 K, or about 900 K.

In some embodiments, the MOₓ catalyst may include a metal oxide having metal centers in cationic form. In some embodiments, the MOₓ catalyst may include at least one of ZrO₂, Y₂O₃, CeO₂, and CoO.

In some embodiments, the MOₓ catalyst may include a metal oxide that is not reducible to zero-valent state.

In some embodiments, the MOₓ catalyst may include a metal (M) selected from the group consisting of Zr, Co, Ga, Zr, Ce, Y, Hf, and Ti. In other embodiments, the MOₓ catalyst may include an oxide of Y, Ce, and Ti.

In some embodiments, the MOₓ catalyst may include one or more of Mg, Ca, Sr, Ba and La on a zirconia support.

In some embodiments, the MOₓ catalyst may include ZrO₂-silica, Zr-alumina, Zr-titania, or a combination thereof.

In some embodiments, the MOₓ catalyst may include ZrO₂, t-ZrO₂, m-ZrO₂, Y-stabilized ZrO₂, Ce-stabilized ZrO₂ or Y₂O₃.

In some embodiments, the method may further include cleaning the MOₓ catalyst with a surface cleaning reagent.

In some embodiments, the surface cleaning reagent possesses all of the following characteristics:
possesses reactivity with one or more bound species derived from CO₂ and/or H₂O via a stoichiometric reaction;
does not lead to one or more reactions that form a surface titrant of a Lewis acid-base pair; and/or
can desorb from a surface of the porous metal oxide catalyst without leaving behind surface debris that can irreversibly titrate an M-O active site of the MOₓ catalyst.

In some embodiments, the surface cleaning reagent is chosen from alcohols, ketones, carboxylates, acids, esters, ethers, hemiacetals, hemiketals, acetals, ketals, orthoesters, orthocarbonates, organic acid anhydrides, and combinations thereof.

In some embodiments, the surface cleaning reagent comprises at least one compound chosen from ROH, RCOR′, RCHO, ROCOOR′, RCOOH, RCOOR′, R₂CH(OR¹)(OH), RC(OR″)(OH)R′, RCH(OR′)(OR″), RC(OR″)(OR″)R′, RC(OR′)(OR″)(OR″), C(OR)(OR′)(OR″)(OR″), and R₁(CO)O(CO)R₂, wherein each of R, R′, R″, R″, R₁, and R₂ is independently chosen from alkyl, alkenyl, alkynyl, and aryl groups (e.g., C₁-C₆ alkyl groups; C₁-C₄ alkyl groups; C₆-C₁₀ aryl groups).

In some embodiments, each of R, R′, R″, R″, R₁, and R₂ is chosen from methyl, phenyl, and tert-butyl.

In some embodiments, R, R′, R″, R″, R₁, and/or R₂ do not possess a —CH₂CH₃ group. In some embodiments, R, R′, R″, R″, R₁, and/or R₂ do not possess a —CH₂CH₃ pendant group. In some embodiments, R, R′, R″, R″, R₁, and/or R₂ do not possess a —CH₂CH₃ terminal group.

In some embodiments, the surface cleaning reagent may include dimethyl ether, propylene, methanol, tert-butyl alcohol, methyl tert-butyl ether, di-tert-butyl ether, anisole, dimethyl carbonate, or combinations thereof. In another embodiment, the surface cleaning reagent may include dimethyl ether. In yet another embodiment, the surface cleaning reagent may include methanol. In some embodiments, the surface cleaning reagent may include an alkene. In another embodiment, the surface cleaning reagent may include propylene.

In some embodiments, the method may further include cleaning the MOₓ catalyst during the dehydrogenation or hydrogenation reaction by balancing water deposition and water removal by the alkene products at a temperature of about 800-900 K.

In some embodiments, the cleaning may be performed at a temperature between about 323 K and 900 K. In some embodiments, the cleaning may be performed at a temperature of about 323 K, about 373 K, about 423 K, about 473 K, about 523 K, about 573 K, about 623K, about 673 K, about 723 K, about 773 K, about 823 K, about 873 K or about 900 K.

In some embodiments, the surface cleaning reagent can desorb from a surface of the metal oxide catalyst without leaving behind surface debris that can irreversibly titrate an M-O active site of the MOₓ catalyst.

In some embodiments, the surface cleaning reagent possesses reactivity with one or more bound species derived from CO₂ and/or H₂O via a stoichiometric reaction; and the surface cleaning reagent does not lead to one or more reactions that form a surface titrant of a Lewis acid-base pair.

In some embodiments, the surface cleaning reagent possesses reactivity with one or more bound species derived from CO₂ and/or H₂O via a stoichiometric reaction; and the surface cleaning reagent and/or its reaction products can desorb from a surface of the MOₓ catalyst without leaving behind surface debris that can irreversibly titrate the M-O active sites in the MOₓ catalyst.

In some embodiments, the surface cleaning reagent possesses reactivity with one or more bound species derived from CO₂ and/or H₂O via a stoichiometric reaction.

In some embodiments, the surface cleaning reagent does not lead to one or more reactions that form a surface titrant of a Lewis acid-base pair.

In some embodiments, the surface cleaning reagent does not lead to one or more reactions that form a surface titrant of a Lewis acid-base pair; and the surface cleaning reagent can desorb from a surface of the MOₓ catalyst without leaving behind surface debris that can irreversibly titrate an M-O active site of the MOₓ catalyst.

In some embodiments, the method further comprises an additional pretreating the catalyst composition in an aerobic oxidative environment before or after the cleaning with the surface cleaning reagent.

In some embodiments, the method further comprises an additional pretreating the catalyst composition in an aerobic oxidative environment before the cleaning with the surface cleaning reagent.

In some embodiments, the method further comprises an additional treatment of the catalyst composition in an aerobic oxidative environment or an anaerobic environment after the cleaning with the surface cleaning reagent.

In another embodiment of the present disclosure, a method of catalyzing a reaction using a catalyst composition as described herein is also provided. In an embodiment, a method of catalyzing a reaction using a catalyst composition including a MOₓ catalyst, including activating and/or reactivating the catalyst composition using the method of pretreating described herein. The reaction may be selected from the group consisting of alkane dehydrogenation, alkene hydrogenation, olefin-paraffin alkylation, from CO/H₂ mixtures without O-rejection as H₂O or CO₂, C—C bond formation via alkene oligomerization or metathesis, dehydrocyclization (alkanes/alkenes to arenes), dehydrocyclodimerization (alkanes/alkenes to arenes with a larger number of C-atoms), transfer hydrogenations, hydroformylation/carbonylation, aromatization, dearomatization, reforming, isomerization, and bifunctional reactions in which one of the aforementioned functions can be optionally combined with a Brønsted acid function.

In some embodiments, the treating of the reactant stream with the trap may be performed simultaneously with the catalyzed reaction. In some embodiments, the treating of reactant stream with the trap may be performed in the same reactor system where the reaction takes place.

In some embodiments, the method further comprises activating and/or reactivating using a method described herein more than once. In some embodiments, the method may further comprise cofeeding a surface cleaning reagent as described herein.

In some embodiments, the bifunctional reaction carried out when the aforementioned functions are optionally combined with a Brønsted acid function is chosen from catalytic reforming for octane enhancement, alkane hydroisomerization, and hydrocracking.

In some embodiments, the bifunctional reaction is chosen from hydroisomerization, hydrocracking, fluid catalytic cracking, and reactions converting C₂-C4 alkanes to aromatics.

In some embodiments, the reaction is alkane dehydrogenation. In some embodiments, the method further comprises cycling between actively dehydrogenating the light alkane gas or light alkene gas with the catalyst composition and reactivating the catalyst composition. In some embodiments, the method further comprises cycling between actively dehydrogenating the light alkane gas or light alkene gas with the catalyst composition and reactivating the catalyst composition. In some embodiments, the method is performed using a plurality of reactors in which the reaction and the activation and/or reactivation of the catalyst are performed alternately.

In some embodiments, the reaction is propane dehydrogenation.

In some embodiments, the reaction occurs in a reactor. In some embodiments, the reactor is chosen from U-shape quartz reactors, packed tubular reactors, fluidized bed reactors, circulating fluidized bed reactors, fixed bed reactors, cycled fixed bed reactors, multi-tubular reactors, cycled sets of multi-tubular reactors and a moving bed reactor, and reactor systems comprising combinations thereof.

In some embodiments, the MOₓ catalyst possesses a surface with an M-O site of the Lewis type and of balanced acid-base strength.

In some embodiments, a surface of the MOₓ catalyst stabilizes anionic and/or cationic moieties that form at transition states for heterolytic processes that form and cleave C—H bonds.

In some embodiments, a metal (M) of the MOₓ catalyst does not undergo reduction to a lower oxidation state in a reductive environment typical of hydrogenation-dehydrogenation catalysis.

In some embodiments, the MOₓ catalyst possesses a surface with an M-O site of the Lewis type and of balanced acid-base strength; a surface of the porous metal oxide catalyst stabilizes anionic and/or cationic moieties that form at transition states for heterolytic processes that form and cleave C—H bonds; and a metal (M) of the MOₓ catalyst does not undergo reduction to a lower oxidation state in a reductive environment typical of hydrogenation-dehydrogenation catalysis.

In some embodiments, the catalyst composition improves product yield compared with a comparable reaction without reactivation.

In some embodiments, the catalyst composition improves the rate of formation, the yield, or the selectivity of one or more desired products relative to the same reaction performed with the catalyst composition without the activating and/or reactivating.

In some embodiments, the reaction may be alkane dehydrogenation. In another embodiment, the reaction may be alkene hydrogenation.

In some embodiments, the MOₓ catalyst may include a metal oxide having metal centers in cationic form.

In some embodiments, the MOₓ catalyst may include a metal oxide that is not reducible to zero-valent state.

In some embodiments, the MOₓ catalyst may include a metal selected from the group consisting of Zr, Co, Ga, Zn, Ce, Y, Hf, and Ti.

In some embodiments, the catalyst composition as described herein including a MOₓ catalyst and trap may improve product yield at least 2-fold compared with a comparable reaction using a catalyst composition that does not include a trap. In some embodiments, the catalyst composition may improve product yield at least 2-fold, at least 5-fold, at least 10-fold, at least 15-fold, at least 20-fold, at least 25-fold, at least 30-fold, at least 35-fold, at least 40-fold, at least 45-fold, at least 50-fold at least 55-fold, at least 60-fold, at least 65-fold, at least 70-fold, at least 75-fold, at least 80-fold, at least 85-fold, at least 90-fold, at least 95-fold, at least 100-fold, at least 110-fold, at least 120-fold, at least 130-fold, at least 140-fold, or at least 150-fold compared with a comparable reaction using a catalyst composition that does not include a trap of the present disclosure. That is, the catalyst composition of the present disclosure having a high surface and active site density as described herein may have an improved product yield.

In some embodiments, the method may further include cycling between dehydrogenating the light alkane gas or hydrogenating the light alkene gas with the catalyst composition and reactivating the catalyst.

In another embodiment, a catalyst composition is provided. In an embodiment, the catalyst composition may include a MOₓ catalyst having a surface with an M-O site of the Lewis type and of balanced acid-base strength and a BET surface area of at least about 50 m²/gram, wherein the catalyst composition may be free of at least one of chromium or a precious metal. In some embodiments, the BET surface area may be at least about 50 m²/gram, at least about 60 m²/gram, at least about 70 m²/gram, at least about 80 m²/gram, at least about 90 m²/gram, or at least about 100 m²/gram.

In some embodiments, the MOₓ catalyst may include a metal oxide having metal centers in cationic form.

In some embodiments, the MOₓ catalyst may include a metal oxide that is not reducible to zero-valent state.

In some embodiments, the MOₓ catalyst may include a metal selected from the group consisting of Zr, Co, Ga, Zn, Ce, Y, Hf, and Ti.

In some embodiments of the catalyst composition, the MOₓ catalyst may include ZrO₂. In some embodiments, the catalyst composition may include at least about 25 wt % ZrO₂ based on total weight of the catalyst composition. In some embodiments, the catalyst composition may include about 25 wt % ZrO₂, about 30 wt % ZrO₂, about 35 wt % ZrO₂, about 40 wt % ZrO₂, about 45 wt % ZrO₂, about 50 wt % ZrO₂, about 55 wt % ZrO₂, or about 60 wt % ZrO₂, or greater.

In some embodiments, the catalyst composition may further include a rare earth metal comprising at least one lanthanide metal, an oxide thereof, or combinations thereof. In another embodiment, the catalyst composition may include a rare earth metal comprising at least one of Y, Er, Ce, Dy, Gd, La, Nd, Sm, Yb, oxides thereof or mixtures thereof.

In some embodiments, the catalyst composition may include about 0.5 wt % to about 50 wt % of the rare earth metal. In some embodiments, the catalyst composition may include about 0.5 wt %, about 1 wt %, about 5 wt %, about 10 wt %, about 15 wt %, about 20 wt %, about 25 wt %, about 30 wt %, about 35 wt %, about 40 wt %, about 45 wt %, or about 50 wt % of the rare earth metal.

In some embodiments, the catalyst composition may be pretreated and may include more surface-active sites than before pretreatment.

In some embodiments, the pretreatment of the reactants may include a trap, wherein the trap removes an impurity. The impurity may include O₂, H₂O, H₂S, CO₂, CO, N₂, a sulfur containing compound, methanol, a ketone, an inorganic nitrogen compound, an organic nitrogen compound, an oxygenate, or a combination thereof. In some embodiments, when treating the reactant stream with a trap of the present disclosure, the reactant stream may be impurity free. In another embodiment, when treating the reactant stream with a trap of the present disclosure, the reactant stream may be substantially free from an impurity.

Some embodiments of this disclosure relate to a method of activating and/or reactivating a catalyst composition comprising a MOₓ catalyst, the method comprising cleaning the catalyst composition with a surface cleaning reagent, wherein:
the surface cleaning reagent is chosen from alcohols, ketones, carboxylates, acids, esters, ethers, hemiacetals, hemiketals, acetals, ketals, orthoesters, orthocarbonates, organic acid anhydrides, and combinations thereof; and further wherein:
if the MOₓ catalyst is ZrO₂, then the surface cleaning reagent is not dimethyl ether or propylene; and
treating the MOₓ catalyst with a trap, wherein the trap may remove an impurity. The impurity may include O₂, H₂O, H₂S, CO₂, CO, N₂, a sulfur containing compound, methanol, a ketone, an inorganic nitrogen compound, an organic nitrogen compound, an oxygenate, or a combination thereof. The treated MOₓ catalyst may have a surface with an M-O site of the Lewis type and of balanced acid-base strength. The treated MOₓ catalyst may have a BET surface area of at least about 100 m²/gram.

In some embodiments, if the MOₓ catalyst is ZrO₂, then the surface cleaning reagent is not methanol.

In some embodiments, the MOₓ catalyst has been rendered inactive by bound H₂O and/or CO₂. In some embodiments, the MOₓ catalyst has been rendered inactive by strongly bound H₂O and/or CO₂.

In some embodiments, the surface cleaning reagent possesses reactivity with one or more bound species derived from CO₂ and/or H₂O via a stoichiometric reaction.

In some embodiments, the surface cleaning reagent does not lead to one or more reactions that form a surface titrant of a Lewis acid-base pair.

In some embodiments, the surface cleaning reagent can desorb from a surface of the porous metal oxide catalyst without leaving behind surface debris that can irreversibly titrate an M-O active site of the porous metal oxide catalyst.

In some embodiments, the surface cleaning reagent possesses reactivity with one or more bound species derived from CO₂ and/or H₂O via a stoichiometric reaction; and the surface cleaning reagent does not lead to one or more reactions that form a surface titrant of a Lewis acid-base pair.

In some embodiments, the surface cleaning reagent possesses reactivity with one or more bound species derived from CO₂ and/or H₂O via a stoichiometric reaction; and the surface cleaning reagent can desorb from a surface of the MOₓ catalyst without leaving behind surface debris that can irreversibly titrate an M-O active site of the porous metal oxide catalyst.

In some embodiments, the surface cleaning reagent does not lead to one or more reactions that form a surface titrant of a Lewis acid-base pair; and the surface cleaning reagent can desorb from a surface of the MOₓ catalyst without leaving behind surface debris that can irreversibly titrate an M-O active site of the porous metal oxide catalyst.

In some embodiments, the surface cleaning reagent is chosen from dimethyl ether, propylene, alkene, methanol, anisole, tert-butyl alcohol, methyl tert-butyl ether, di-tert-butyl ether, dimethyl carbonate, and combinations thereof.

In some embodiments, the surface cleaning reagent is chosen from dimethyl ether, propylene, and methanol.

In some embodiments, the surface cleaning reagent is dimethyl ether.

In some embodiments, the surface cleaning reagent is propylene.

In some embodiments, the surface cleaning reagent is methanol.

In some embodiments, the MOₓ catalyst possesses a surface with an M-O site of the Lewis type and of balanced acid-base strength.

In some embodiments, a surface of the MOₓ catalyst stabilizes anionic and/or cationic moieties that form at transition states for heterolytic processes that form and cleave C—H bonds.

In some embodiments, a metal (M) of the MOₓ catalyst does not undergo reduction to a lower oxidation state in a reductive environment typical of hydrogenation-dehydrogenation catalysis.

In some embodiments, the MOₓ catalyst possesses a surface with an M-O site of the Lewis type and of balanced acid-base strength; a surface of the MOₓ catalyst stabilizes anionic and/or cationic moieties that form at transition states for heterolytic processes that form and cleave C—H bonds; and a metal (M) of the MOₓ catalyst does not undergo reduction to a lower oxidation state in a reductive environment typical of hydrogenation-dehydrogenation catalysis.

In some embodiments, the MOₓ catalyst may include a metal oxide having metal centers in cationic form.

In some embodiments, the MOₓ catalyst may include a metal oxide that is not reducible to zero-valent state.

In some embodiments, the MOₓ catalyst may include a metal selected from the group consisting of Zr, Co, Ga, Zn, Ce, Y, and Ti.

In some embodiments of the catalyst composition, the MOₓ catalyst may include ZrO₂. In some embodiments, the catalyst composition may include at least about 50 wt % ZrO₂ based on total weight of the catalyst composition.

In some embodiments, the MOₓ catalyst may include Y-stabilized ZrO₂. In some embodiments, the MOₓ catalyst is Y-stabilized ZrO₂.

In some embodiments, the MOₓ catalyst may include Y₂O₃. In some embodiments, the MOₓ catalyst is Y₂O₃.

In some embodiments, the cleaning may be performed at a temperature of up to 900 K. In some embodiments, the cleaning may be performed at a temperature of up to 873 K. In some embodiments, the cleaning may be performed at a temperature of up to 823 K. In some embodiments, the cleaning may be performed at a temperature of up to 723 K.

In some embodiments, the cleaning may be performed at a temperature between 323 K and 900 K. In some embodiments, the cleaning may be performed at a temperature between 323 K and 873 K. In some embodiments, the cleaning may be performed at a temperature between 323 K and 823 K. In some embodiments, the cleaning may be performed at a temperature between 323 K and 723 K.

In some embodiments, the method may further include an additional pretreating the catalyst composition in an aerobic oxidative environment before or after the pretreatment with the surface cleaning reagent.

In some embodiments, the method may further include pretreating the catalyst composition in an aerobic oxidative environment before the pretreatment with the surface cleaning reagent.

In some embodiments, the method may further include pretreating the catalyst composition in an aerobic oxidative environment after the pretreatment with the surface cleaning reagent.

Some embodiments of this disclosure relate to a method of activating and/or reactivating a catalyst composition comprising a MOₓ catalyst and a trap, the method comprising cleaning the catalyst composition with a surface cleaning reagent, wherein:

the surface cleaning reagent comprises at least one compound chosen from ROH, RCOR′, RCHO, ROCOOR′, RCOOH, RCOOR′, R₂CH(OR₁)(OH), RC(OR″)(OH)R′, RCH(OR′)(OR″), RC(OR″)(OR″)R′, RC(OR′)(OR″)(OR″), C(OR)(OR′)(OR″)(OR″), and R₁(CO)O(CO)R₂, wherein each of R, R′, R″, R″, R₁, and R₂ is independently chosen from alkyl, alkenyl, alkynyl, and aryl groups (e.g., C₁-C₆ alkyl groups; C₁-C₄ alkyl groups; C₆-C₁₀ aryl groups); and further wherein:
if the MOₓ catalyst is ZrO₂, then the surface cleaning reagent is not dimethyl ether or propylene; and
treating the reactant stream with a trap, wherein the trap may remove an impurity. The impurity may include O₂, H₂O, H₂S, CO₂, CO, N₂, a sulfur containing compound, methanol, a ketone, an inorganic nitrogen compound, an organic nitrogen compound, an oxygenate, or a combination thereof. The treated MOₓ catalyst may have a surface with an M-O site of the Lewis type and of balanced acid-base strength. The treated MOₓ catalyst may have a BET surface area of at least about 75 m²/gram.

In some embodiments, if the MOₓ catalyst is ZrO₂, then the surface cleaning reagent is not methanol.

In some embodiments, the MOₓ catalyst has been rendered inactive by bound H₂O and/or CO₂. In some embodiments, the MOₓ catalyst has been rendered inactive by strongly bound H₂O and/or CO₂.

In some embodiments, each of R, R′, R″, R″, R₁, and R₂ is chosen from methyl, phenyl, and tert-butyl.

In some embodiments, R, R′, R″, R″, R₁, and/or R₂ do not possess a —CH₂CH₃ group. In some embodiments, R, R′, R″, R″, R₁, and/or R₂ do not possess a —CH₂CH₃ pendant group. In some embodiments, R, R′, R″, R″, R₁, and/or R₂ do not possess a —CH₂CH₃ terminal group.

In some embodiments, the surface cleaning reagent possesses reactivity with one or more bound species derived from CO₂ and/or H₂O via a stoichiometric reaction.

In some embodiments, the surface cleaning reagent does not lead to one or more reactions that form a surface titrant of a Lewis acid-base pair.

In some embodiments, the surface cleaning reagent can desorb from a surface of the porous metal oxide catalyst without leaving behind surface debris that can irreversibly titrate an M-O active site of the MOₓ catalyst.

In some embodiments, the surface cleaning reagent possesses reactivity with one or more bound species derived from CO₂ and/or H₂O via a stoichiometric reaction; and the surface cleaning reagent does not lead to one or more reactions that form a surface titrant of a Lewis acid-base pair.

In some embodiments, the surface cleaning reagent possesses reactivity with one or more bound species derived from CO₂ and/or H₂O via a stoichiometric reaction; and the surface cleaning reagent can desorb from a surface of the MOₓ catalyst without leaving behind surface debris that can irreversibly titrate an M-O active site of the MOₓ catalyst.

In some embodiments, the surface cleaning reagent does not lead to one or more reactions that form a surface titrant of a Lewis acid-base pair; and the surface cleaning reagent can desorb from a surface of the MOₓ catalyst without leaving behind surface debris that can irreversibly titrate an M-O active site of the MOₓ catalyst.

In some embodiments, the surface cleaning reagent is chosen from dimethyl ether, propylene, methanol, anisole, tert-butyl alcohol, methyl tert-butyl ether, di-tert-butyl ether, dimethyl carbonate, and combinations thereof.

In some embodiments, the surface cleaning reagent is chosen from dimethyl ether, propylene, and methanol.

In some embodiments, the surface cleaning reagent is dimethyl ether.

In some embodiments, the surface cleaning reagent is propylene.

In some embodiments, the surface cleaning reagent is methanol.

In some embodiments, the MOₓ catalyst possesses a surface with an M-O site of the Lewis type and of balanced acid-base strength.

In some embodiments, a surface of the MOₓ catalyst stabilizes anionic and/or cationic moieties that form at transition states for heterolytic processes that form and cleave C—H bonds.

In some embodiments, a metal (M) of the MOₓ catalyst does not undergo reduction to a lower oxidation state in a reductive environment typical of hydrogenation-dehydrogenation catalysis.

In some embodiments, the MOₓ catalyst possesses a surface with an M-O site of the Lewis type and of balanced acid-base strength; a surface of the MOₓ catalyst stabilizes anionic and/or cationic moieties that form at transition states for heterolytic processes that form and cleave C—H bonds; and a metal (M) of the MOₓ catalyst does not undergo reduction to a lower oxidation state in a reductive environment typical of hydrogenation-dehydrogenation catalysis.

In some embodiments, the MOₓ catalyst may include a metal oxide having metal centers in cationic form.

In some embodiments, the MOₓ catalyst may include a metal oxide that is not reducible to zero-valent state.

In some embodiments, the MOₓ catalyst may include a metal selected from the group consisting of Zr, Co, Ga, Zn, Ce, Y, and Ti.

In some embodiments of the catalyst composition, the MOₓ catalyst may include ZrO₂. In some embodiments, the catalyst composition may include at least about 25 wt % ZrO₂ based on total weight of the catalyst composition.

In some embodiments, the MOₓ catalyst may include Y-stabilized ZrO₂. In some embodiments, the MOₓ catalyst is Y-stabilized ZrO₂.

In some embodiments, the MOₓ catalyst may include Y₂O₃. In some embodiments, the MOₓ catalyst is Y₂O₃.

In some embodiments, the cleaning may be performed at a temperature of up to 900 K. In some embodiments, the cleaning may be performed at a temperature of up to 873 K. In some embodiments, the cleaning may be performed at a temperature of up to 823 K. In some embodiments, the cleaning may be performed at a temperature of up to 723 K.

In some embodiments, the cleaning may be performed at a temperature between 323 K and 900 K. In some embodiments, the cleaning may be performed at a temperature between 323 K and 873 K. In some embodiments, the cleaning may be performed at a temperature between 323 K and 823 K. In some embodiments, the cleaning may be performed at a temperature between 323 K and 723 K.

In some embodiments, the method may further include pretreating the catalyst composition in an aerobic oxidative environment before or after the cleaning with the surface cleaning reagent and treating the reactants with a trap.

In some embodiments, the method may further include pretreating the catalyst composition in an aerobic oxidative environment before the cleaning with the surface cleaning reagent and treating the reactants with a trap.

In some embodiments, the method may further include pretreating the catalyst composition in an aerobic oxidative environment after the cleaning with the surface cleaning reagent and treating the reactants with a trap.

Claims or descriptions that include “or” or “and/or” between at least one members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Furthermore, the disclosure encompasses all variations, combinations, and permutations in which at least one limitation, element, clause, and descriptive term from at least one of the listed claims is introduced into another claim. For example, any claim that is dependent on another claim can be modified to include at least one limitation found in any other claim that is dependent on the same base claim. Where elements are presented as lists, such as, e.g., in Markush group format, each subgroup of the elements is also disclosed, and any element(s) can be removed from the group. It should be understood that, in general, where the disclosure, or aspects of the disclosure, is/are referred to as comprising particular elements and/or features, embodiments of the disclosure or aspects of the disclosure consist, or consist essentially of, such elements and/or features. For purposes of simplicity, those embodiments have not been specifically set forth in haec verba herein. Where ranges are given (such as, e.g., from [X] to [Y]), endpoints (such as, e.g., [X] and [Y] in the phrase “from [X] to [Y]”) are included unless otherwise indicated. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those of ordinary skill in the art will recognize or be able to ascertain using no more than routine experimentation, many equivalents to the specific embodiments of the disclosure described herein. Such equivalents are intended to be encompassed by the following claims.

### EXAMPLES

The following examples are intended to be illustrative and are not meant in any way to limit the scope of the disclosure.

Several tests were conducted to test various materials for dehydrogenation reactions that could benefit from thermal or chemical treatments and may remove impurities from the stream and from the surface of the catalysts. Tests were also conducted to measure the reaction rates as one of the means to assess the benefits of such treatments.

### Example 1a: Monoclinic-ZrO₂ (m-ZrO₂)

Monoclinic-ZrO₂ (m-ZrO₂) was prepared using hydrothermal synthesis methods using the decomposition of urea to gradually increase the solution pH and induce precipitation as described in Li, W. et al. “Facile Synthesis of Pure Monoclinic and Tetragonal Zirconia Nanoparticles and Their Phase Effects on the Behavior of Supported Molybdena Catalysts for Methanol Selective Oxidation” Langmuir 2008, 8358-8366. ZrO(NO₃)₂·xH₂O (12.7 g; Sigma-Aldrich) and CO(NH₂)₂ (21.6 g; Sigma-Aldrich) were each dissolved in deionized water (≥17.6 MΩ-cm resistivity; 30 g) at 323 K. The two solutions were mixed in a Teflon-lined autoclave (Parr, 125 cm³) and held at 393 K for 20 h. The powders formed were rinsed with deionized water (250 g) and centrifuged four times followed by treatment in ambient air at 393 K for 12 h. These samples were then treated in flowing He (Praxair, 99.999%, 1.6 cm³ g⁻¹ s⁻¹) at 723 K for 5 h.

### Example 1b: Tetragonal-ZrO₂ (t-ZrO₂)

Tetragonal-ZrO₂ (t-ZrO₂) was prepared using methanothermal synthesis methods as described in Li, W. et al. “Facile Synthesis of Pure Monoclinic and Tetragonal Zirconia Nanoparticles and Their Phase Effects on the Behavior of Supported Molybdena Catalysts for Methanol Selective Oxidation” Langmuir 2008, 8358-8366. ZrO(NO₃)₂·xH₂O (6.35 g; Sigma-Aldrich) and CO(NH₂)₂ (10.8 g; Sigma-Aldrich) were each dissolved in methanol (Fisher Scientific, 99.9%, 30 g) at 323 K. The two solutions were mixed in a Teflon-lined autoclave (Parr, 125 cm³) and held at 393 K for 20 h. The powders formed were washed with methanol (100 g) followed by treatment in ambient air at 393 K for 12 h. These samples were then treated in flowing air (Praxair, 99.999%, 1.6 cm3 g⁻¹ s⁻¹) at 723 K for 5 h.

### Example 2: Y—ZrO₂

Y—ZrO₂ was prepared by dissolving ZrOCl₂·8H₂O (Sigma-Aldrich) and Y(NO₃)₃·6H₂O starting materials in deionized water (≥17.6 MΩ-cm resistivity) to form a 125 ml solution (C_{Zr}=0.2 mol L⁻¹) in which Y³⁺ and ZrO²⁺ concentrations correspond to 1:9 molar ratio as described in Huang et al. “The Effects of Precipitants on Co-Precipitation Synthesis of Yttria-Stabilized Zirconia Nanocrystalline Powders” J. Sol-Gel Sci. Tech. 2019, 90, 359-368. NH₄OH (13 ml, 25-28 wt % in water) was added into ethanol-water solution (200 ml ethanol and 800 ml water) to form the precipitation solution, after which the main solution was added dropwise into the precipitation solution at room temperature and stirred with magnetic stirrer for 1 h, followed by the aging for 6 h. The powders formed were rinsed with deionized water (250 g) and ethanol (250 g) and centrifuged four times followed by treatment in ambient air at 333 K for 1 h. These samples were then treated in flowing He (Praxair, 99.999%, 1.6 cm3 g⁻¹ s⁻¹) at 723 K for 2 h.

### Example 3: Supported CoO materials

Silica-supported cobalt catalysts are known active materials for alkane dehydrogenation reactions. The support can be tailored to accommodate better dispersed CoO domains by introducing additional anchoring sites (e.g. hydroxyl groups). SiOₓ(OH)₄₋₂ₓ supports (301 m² g⁻¹) were prepared by rehydroxylation of SiO₂ (293 m² g⁻¹) (˜10 g) in a stirring aqueous solution of pH 2-5, set by adding 2 N HNO₃ for 18 h at 373 K. The resulting aqueous slurry of SiOₓ(OH)₄₋₂ₓ (˜12 g) was thoroughly washed by distilled water (100 cm³ g⁻¹) and then dried at 393 K for 12 h before subsequent treatments to prepare the catalyst. Supported CoOx catalysts were prepared by impregnating SiOx(OH)₄₋₂ₓ (301 m² g⁻¹) with drops of an aqueous solution (˜0.25 cm₃ H₂O per g support) of cobalt (II) acetate tetrahydrate to the point of incipient wetness. Samples were dried in a tray at 393 K in ambient air for 9 h and treated in flowing dry air (1.67 cm³ s⁻¹) at 923 K for 3 h to get 5.0-7.5 wt. % and 0.3-20 wt. % Co-catalysts.

### Example 4: Representative Reaction Rate Measurement

Propane (Praxair, 49.7±2%, balance Ar) dehydrogenation (PDH) rates were measured in the presence of H₂ (Praxair, 99.999%) and He (Praxair, 99.999%) on sieved catalyst particles (177-250 μm) held within a U-shape quartz reactor (10 mm i.d.). Catalyst particles were diluted with quartz sand (Sigma Aldrich, 210-297 μm, treated in air, 1073 K, 8 h), in 1:50 mass ratios to circumvent potential corruptions in measured rates caused by bed-scale temperature gradients. Prior to PDH rate measurements, catalyst beds were treated in flowing He (Praxair, 99.999%, 0.83 cm³ g⁻¹ s⁻¹) at 723 K (heating rate 0.4 K s⁻¹) for 3.6 ks. Dimethyl ether (DME) treatments optionally take place to cleanse surface sites. Such treatments are comprised on flowing about 1 to about 5 kPa DME for 15 minutes through the sample, followed by a period of He purge (e.g. 25 mins), prior to rate measurements.

Additional experiments were conducted in a similar method for ethane, n-butane, and isobutane.

Inlet flow rates were set via electronic mass flow controllers (Porter). The desired mixtures of propane, H₂, and He were introduced using adjusted flows for each gas. The reactant stream was optionally passed through the oxygen trap (Agilent 5182-9401, 5 ppb) installed before the catalytic reactor.

The catalytic bed temperature was measured using a K-type thermocouple and kept constant by using resistive heating and an electronic temperature controller (Watlow 96). Reactant and product concentrations were determined by on-line gas chromatography (Agilent, 6890A, GS-GASPRO capillary column, 15 m, 0.32 mm diameter) using a flame ionization detector (FID). Forward propene formation rates per mass of catalyst (rₘ) were calculated by correcting measured rates for approach to equilibrium (η) by Equations 1a and 1b.

$${r}_{m} = \frac{{F}_{net , {C}_{3} {H}_{6}}}{{m}_{Z r {O}_{2}} ( 1 - η )} \qquad ( Equation 1 a )$$

$$η = \frac{{P}_{{C}_{3} {H}_{6}} {P}_{{H}_{2}}}{{P}_{{C}_{3} {H}_{8}} {P}^{0} {K}_{eq , DH}} \qquad ( Equation 1 b )$$

where F_{net,C}_{₃}_{H}_{₆}, m_{ZrO}_{₂}, and n denote molar flow rate of propene in the reactor effluent, mass of ZrO₂, and approach to equilibrium of non-oxidative dehydrogenation of propane (Equation 1a), respectively; Pᵢ, P⁰, and K_{eq,DH} denote the pressure of species i (i=C₃H₈, H₂, C₃H₆) in units of bars and the equilibrium constant of non-oxidative dehydrogenation of propane, respectively. Dehydrogenation areal rates, rₐ, are defined in Equation 1c as:

$${r}_{a} = \frac{{r}_{m}}{S {A}_{Z r {O}_{2}}} \qquad ( Equation 1 c )$$

where SA_{ZrO}_{₂}, denotes the surface area of ZrO₂ per gram. Dehydrogenation selectivities are reported on a carbon basis, as the rate ratio of propane conversion resulting in propene formation to that leading to the formation of smaller molecules (methane, ethane and ethylene).

To assess the stability and deactivation of catalysts, a first-order deactivation rate constant (k_{d}) was regressed using Equation 1d:

$$\frac{{r}_{a} ( t )}{{r}_{a} ( {t}_{0} )} = exp [ - {k}_{d} ( t - {t}_{0} ) ] \qquad ( Equation 1 d )$$

where rₐ(t) and rₐ(t₀) represent PDH rates at an initial t₀ and at a given time t.

Product selectivities and dehydrogenation rates were determined by subtracting contributions from any gas phase homogenous reactions that can occur in the absence of active catalyst materials. Typical product selectivities on the catalysts were reported being above 99%.

### Example 5: Tests to Show that Small Crystalline Provide the Requisite Combination of High Surface Area and High Areal Site Densities for Use in Dehydrogenation and Hydrogenation Reactions

Thermal treatments can negatively affect surface area and cause sintering and annealing of crystallites, thus decreasing surface areas and the areal density of active sites. It has been found that thermal treatments can also partially activate materials via incomplete dehydroxylation when treatments are not carried out at an elevated temperature of 700 to 750 K.

A ZrO₂ sample as prepared in Example 1a was treated at different temperatures (723-1108 K) in flowing He (Praxair, 99.999%, 1.6 cm³ g⁻¹ s⁻¹) with a heating rate of 0.16 K s⁻¹ and kept at target temperature for 1.8 ks. The surface areas of samples was measured from N₂ uptakes at its normal boiling point (3Flex, Micromeritics). The crystal structure and crystallite size of ZrO₂ were determined by X-ray diffraction (Rigaku Miniflex, Cu-Kα radiation) in the 2θ range of 20-80° with a step scan of 0.01°. The particle size in ZrO₂ samples was calculated from X-ray diffractograms using Rietveld refinement and the Scherrer equation and from surface area values using ZrO₂ density at a given temperature and Sauter mean diameter formula (d₃₂=6Vₚ/Aₚ, where Vₚ and Aₚ are the volume and surface area of the particle).

After measuring dehydrogenation rates on thermally treated ZrO₂, ZrO₂ samples were treated with 1-5 kPa dimethyl ether (DME) (Praxair, 5.07±0.2% dimethyl ether, 5.07±0.2% Ar, balance He) for 0.3-1.8 ks at a temperature range of 523-723 K followed by treatment in flowing He at 723 K for 1.8 ks to fully dehydroxylate the surface and expose the Lewis Acid-Base (LAB) active sites.

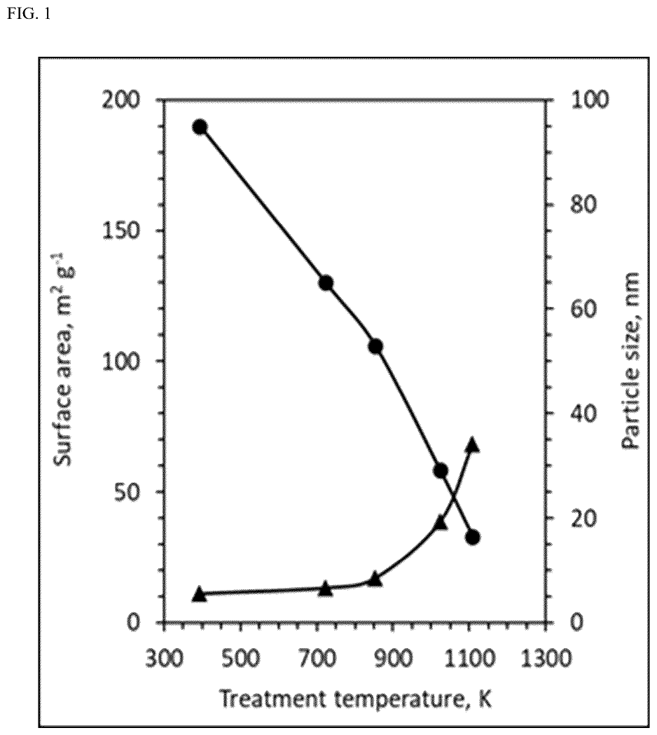

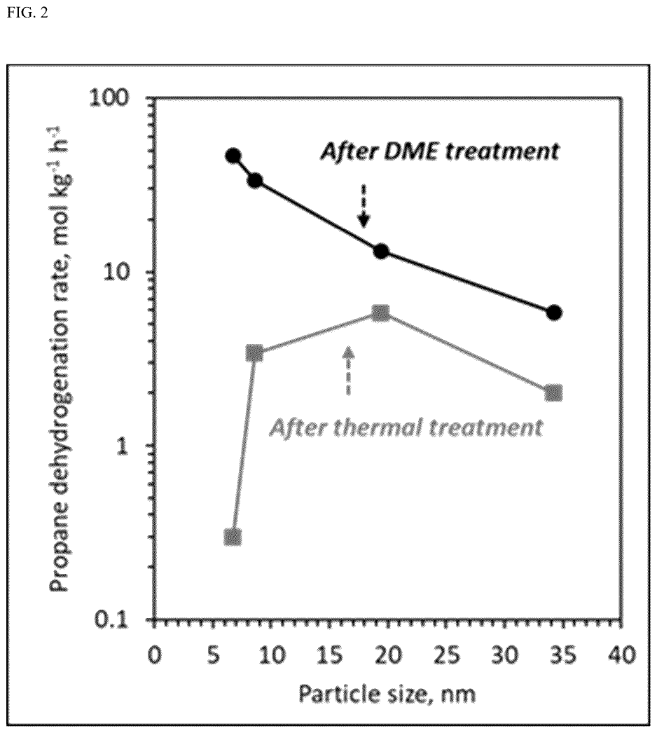

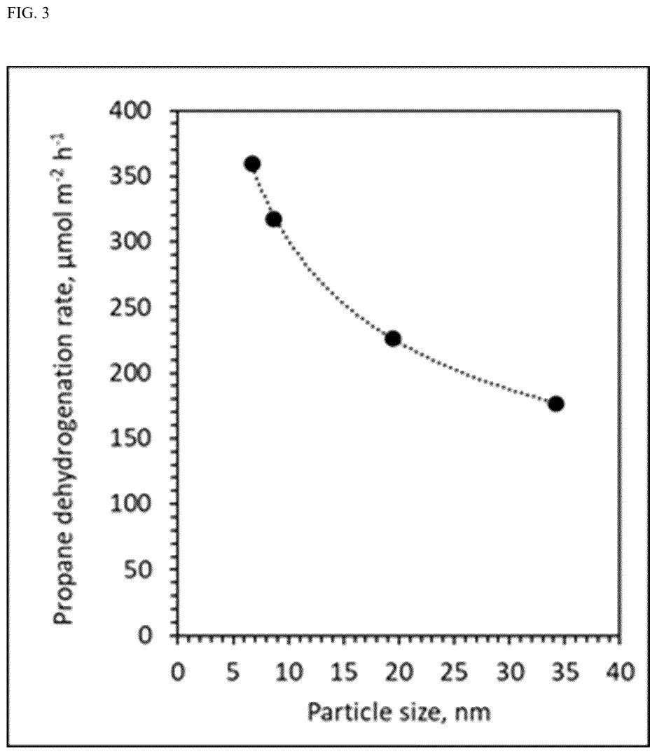

The results of these treatments are shown in the graphs presented in FIGS. 1-3. As can be seen in FIG. 1, there was a decrease in surface area and a concomitant increase in crystallite diameter with increasing thermal treatment temperatures. In FIG. 2, the results show that a thermal treatment caused an initial increase in rates (per mass) as temperature increased, as a result of the counteracting effects of partial dehydroxylation and sintering/annealing. In contrast, when treated with DME, which dehydroxylates the surface, the thermal treatments (before DME cleaning) decreased rates as a result of the concurrent decrease in surface area and areal site density without any additional dehydroxylation, because such hydroxyls were eventually removed by DME cleaning. In FIG. 3, the graph shows the rate per surface area after thermal treatment and DME cleaning demonstrated that even after correcting for the loss of surface area, thermal treatments decreased rates because the number of sites per surface area also decreased because of particle growth and loss of the undercoordinated sites that provide the most catalytically-competent LAB pairs.

### Example 6: Site Titration Methods and Density of Active Centers on Oxide Materials

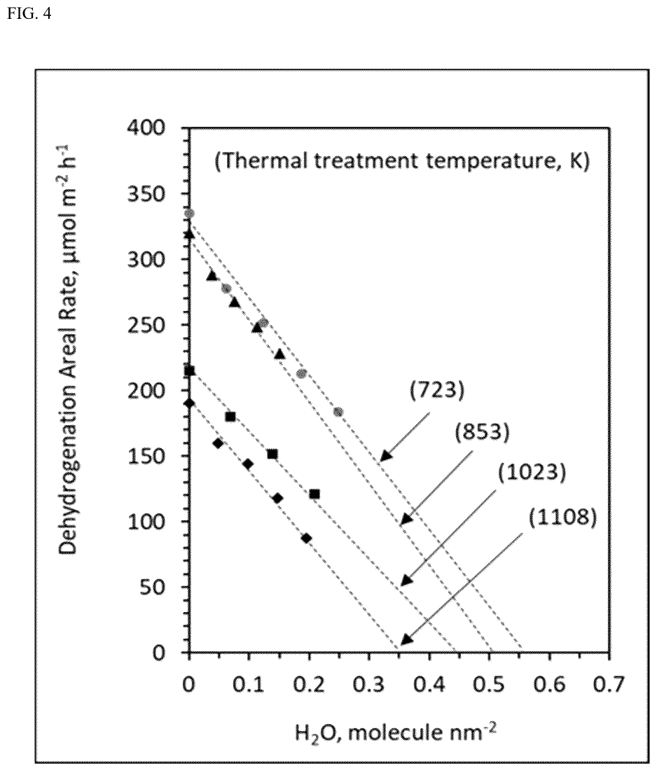

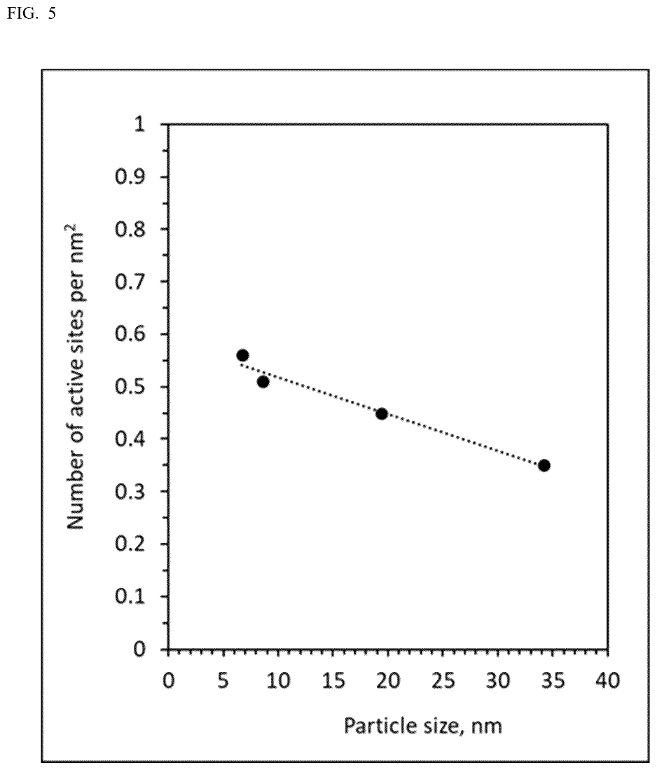

As previously demonstrated, DME treatments remove hydroxyl groups that block active sites. This can be seen in WO 2022/132843 and International Application No. PCT/US2022/077002. The clean active sites exposed by such treatments can then be counted using water as a titrant, which restores hydroxyl groups, rendering the active sites inaccessible to reactants. To determine the number of active sites, H₂O and O₂ titrations were performed during dehydrogenation reaction by pulse injections of 0.61 kPa H₂O or 0.5 kPa O₂ (the latter leading to H₂O formation via reactions with H₂ at reaction conditions) into the reactant stream, using a 1 cm³ loop attached to a 6-way valve (VICI). The results of these protocols and measurements are shown in FIGS. 4 and 5. It should be understood that these are intended as illustrative examples.

In FIG. 4, the graph shows a linear decrease in rates as a function of the amount of water injected with the x-axis intersect defining the number of water molecules required to suppress reactivity, which gives the number of active sites. In FIG. 5, the graph shows that the number of active sites decreases with increasing crystallite diameter. Therefore, the inventors have concluded this demonstrates that the growth of crystallites caused by thermal treatment decreases the surface density of sites as a result of annealing. Therefore, there is a need for small crystallites not only to provide high surface area but also a high areal density of active sites, the product of which ultimately determines the number of active sites per mass of catalyst and thus the mass-based productivity for the reaction catalyzed by such sites

### Example 7. The Beneficial Use of Monoclinic ZrO₂ for High Dehydrogenation Rates

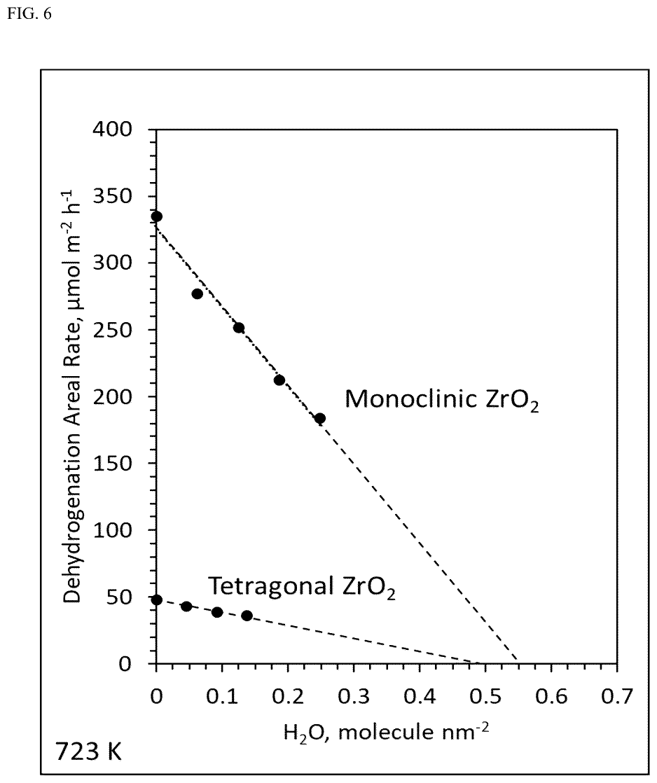

In FIG. 6, the graph shows a linear decrease in rates for pure monoclinic and tetragonal ZrO₂ as a function of the amount of water injected with the x-axis intersect defining the number of water molecules required to suppress reactivity, which provides the number of active sites. In FIG. 6, the graph shows that the number of active sites per surface area is similar for both m-ZrO₂ and t-ZrO₂ catalysts. However, dehydrogenation rate for t-ZrO₂ is about 7 times lower than that measured for m-ZrO₂. Therefore, it can be concluded that that the number of active sites is not the only important feature, but that the structure of the ZrO₂ itself, i.e., the Zr—O coordination environment is also consequential for site-based reactivity. Therefore, for high dehydrogenation rates, a monoclinic structure may be used.

### Example 8: Support of Example 5 Shows Also Beneficial Effects of Small Crystallites for Isobutane Dehydrogenation

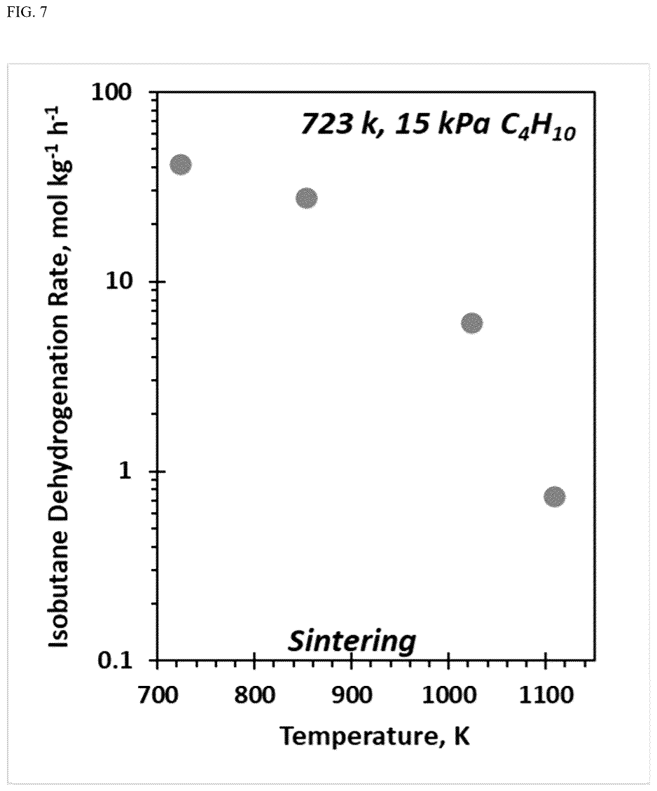

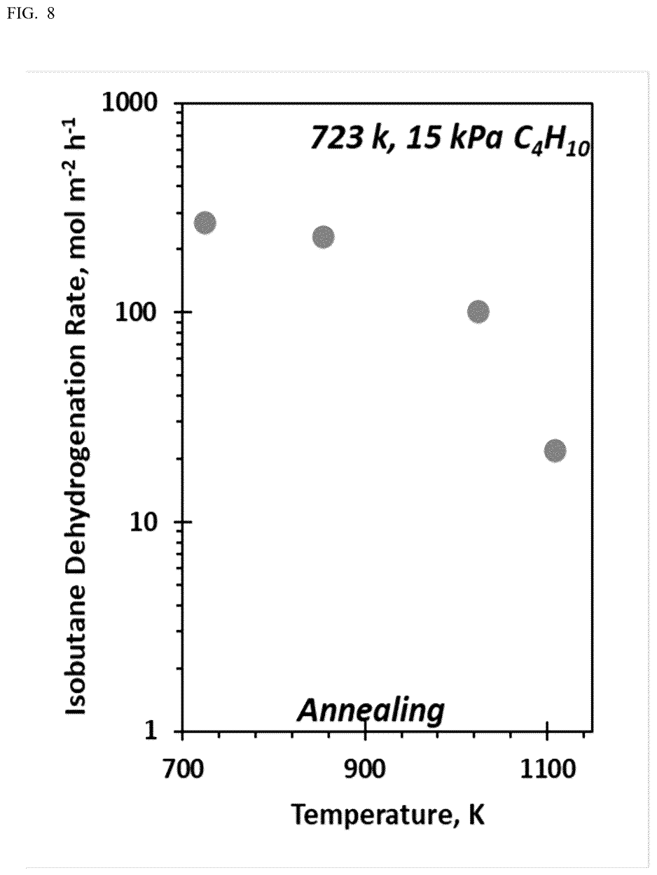

In FIGS. 7 and 8, graphs were prepared showing that similar requirements for small crystallites to provide both high surface areas and site densities are also applicable for isobutane dehydrogenation and not only for propane dehydrogenation, which requires the activation of primary and tertiary C—H bonds instead of primary and secondary C—H bonds in propane. Similarly, to the above examples, a sample of m-ZrO₂ as prepared in Example 1a was treated at different temperatures. The present results show that both the mass-normalized and areal rates decreased with higher temperatures as the material sinters and anneals, resulting in a reduction of both surface area and areal site density with higher temperature thermal treatments. Isobutane dehydrogenation data at the temperatures displayed in FIGS. 7 and 8 represent initial dehydrogenation rates, extrapolated to the point of feed introduction, and are shown for a single batch of catalyst which was subsequently exposed in sequence to each treatment, rather than a fresh sample for each data point.

### Example 9: Changes in Crystallite Size, Surface Area, and Active Site Density with Different Thermal Treatment Temperatures

The changes in crystallite size, surface area, and active site density with different thermal treatment temperatures are presented in Table 1. Table 1 enumerates the nomenclature for the catalysts and their physicochemical properties. Samples A-D were prepared following the steps described in Example 1a. The treatment temperatures of Samples A-D are specified in Table 1. Samples E and F were prepared following the same steps described in Example 2. The treatment temperatures of Samples E and F are also specified in Table 1.

TABLE 1
Surface area, crystallite size and active site density after thermal treatments

| | | | | Calculated | Calculated | | | |
|---|---|---|---|---|---|---|---|---|
| | | | | crystallite | crystallite | Active | Active | Side |
| | | | | diameter | diameter | site | site | phase, % |
| | | Treatment | Surface | (from XRD | (from | density, | density, | (from |
| | | temperature, | area, | patterns), | surface | μmol_{Zr—O} | Zr—O | XRD |
| Sample | Catalyst | K | m² g⁻¹ | nm | area), nm | g⁻¹ | nm⁻² | patterns) |
| Example 1a | m-ZrO₂ | 573 | 190 | 5.6 | 5.9 | — | — | — |
| | | 723 | 130 | 6.7 | 8.7 | 119 | 0.55 | — |
| | | 853 | 106 | 8.6 | 10.6 | 90 | 0.51 | 7% t-ZrO₂ |
| | | 1023 | 58.3 | — | 19.4 | 44 | 0.45 | 19% t-ZrO₂ |
| | | 1108 | 33.1 | — | 34.2 | 19 | 0.35 | 27% t-ZrO₂ |
| Example 2 | Y-ZrO₂ | 573 | 113 | — | 10.7 | — | — | — |
| | (1:9) | 723 | 113 | — | 10.7 | 60 | 0.32 | — |
| | | 923 | 112 | — | — | — | — | — |
| Example 1b | t-ZrO₂ | 573 | 198 | N/A | 5.7 | — | — | — |
| | | | | (amorphous) | | | | |
| | | 723 | 89 | 6.0 | 10.9 | 77 | 0.52 | — |

### Example 10: Catalyst Structural Properties from XRD Patterns after Different Thermal Treatment Temperatures in He and with Yttrium Oxide Addition as a Stabilizer of the Tetragonal Crystal Phase of Zirconia

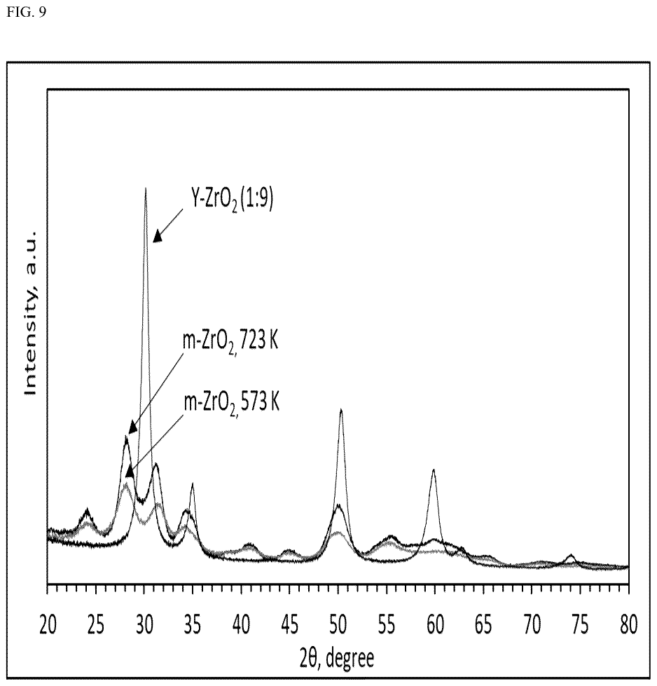

Catalyst structural properties were determined for samples of Examples 1a and 2 as can be seen in the X-ray diffractograms in FIG. 9. The samples of Example 1a were treated at 573 and 723 K, while the sample of Example 2 was treated at 573 K. The thermal treatment was conducted in the presence of He and with yttrium oxide addition as a stabilizer of the tetragonal crystal phase of zirconia.

### Example 11: Methods of Removing Titrant Impurities and Precursors to Titrant Molecules from Inlet Streams to Obtain and Maintain High Alkane Dehydrogenation Rates

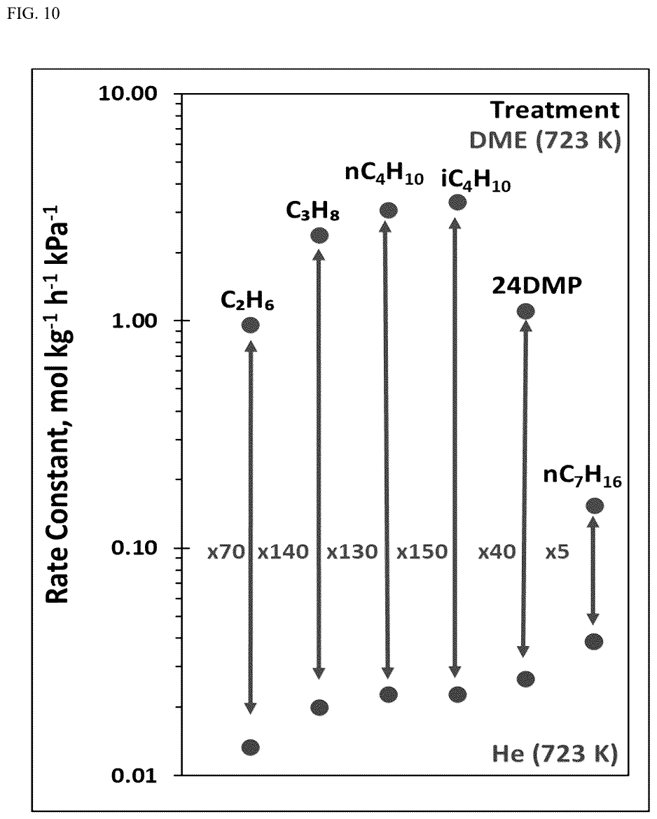

Given that areal dehydrogenation rates for other alkanes also decrease with increasing thermal treatment temperatures, as samples sinter and sites anneal, it was shown that the removal of impurities from reactant streams and thermal treatments and chemical cleaning led to similar effects and trends for the dehydrogenation of other molecules. This is believed to be because the free Lewis acid-base pairs that serve as active centers can also be exposed by DME treatments and feed cleaning protocols for such reactions of other alkanes. The data shown in FIG. 10 represents initial first-order dehydrogenation rate constants (where all rates are proportional to alkane pressure and insensitive to alkene or H₂ pressures), and were then extrapolated to the point of initial alkane feed introduction, after He or DME treatments at 723 K.

As can be seen in FIG. 10, the effects of DME treatment were observed for all alkanes. Consequently, the removal of titrants from surfaces and the prevention of their reintroduction with the reactant stream are essential to obtain and maintain high rates for all reactants. It is further noted that FIG. 10 represents alkane dehydrogenation rate constants for C₂-C₇ alkanes after He and DME treatments at 723 K on samples of Example 1a. The 2,4,-DMP in FIG. 10 denotes 2,4-dimethylpentane. The double head arrows and “x number” in FIG. 10 indicate the rate increase of the catalyst measured after DME treatment. For example, the first double head arrow and “×70” indicates 70 times more active after DME treatment than when subject to only a thermal treatment at the same temperature.

### Example 12: Removing Impurities that Titrate Lewis Acid-Base Pairs and their Precursors

Water- and O₂-derived species act as titrants that cover active sites for catalysis. Oxygen is converted to water during the reaction via reactions with added or produced H₂. Oxygen and water are equally competent molecules to measure the active site density, within the factor of 2 set by the fact that each O₂ molecule produces two H₂O molecules. Both H₂O and CO₂ molecules can also be formed by reaction with propane reactants, or the propene co-produced with H₂.

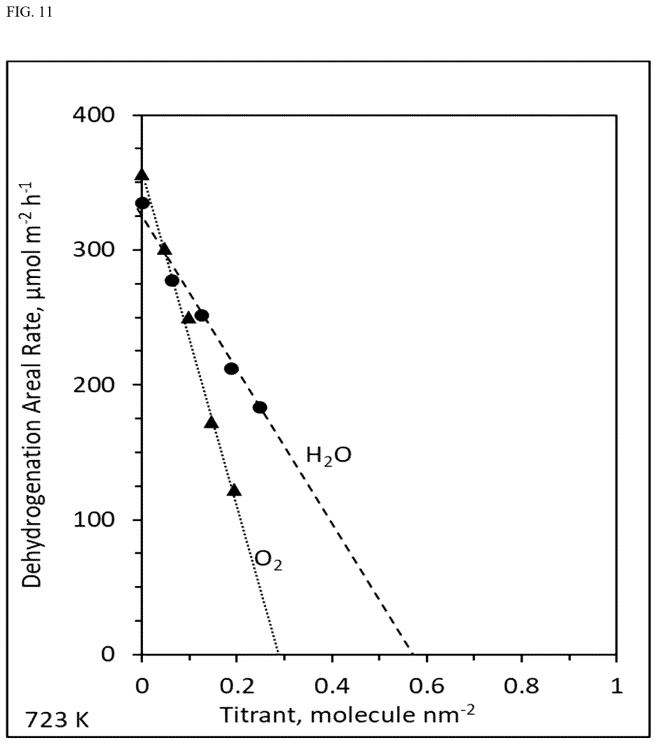

FIG. 11 illustrates the titration of active sites by O₂ and H₂O during propane dehydrogenation catalysis (723 K, 15 kPa, C₃H₈, 5 kPa, H₂), ZrO₂ of Example 1a. FIG. 11 demonstrates that the O₂ and water are both strong irreversible titrants of dehydrogenation-hydrogenation active sites and that each O₂ molecule titrates two active sites because it reacts with H₂ in the inlet stream to form two H₂O molecules. Further, FIG. 11 also demonstrates the need not only to remove water but also any precursors to water (or to any other acid or base molecules that strictly titrate Lewis acid-base pairs). It can also be inferred by these data that other molecules that act as titrants because of their acid-base nature (e.g. CO₂, NH₃, oxygenates) should not only be removed from the feed but that any molecules that can form such molecules via reactions with H₂, alkanes, alkenes, contact with the catalyst, or thermal decomposition also be removed before any stream contacts the catalyst.

### Example 13: Use of a Trap to Remove Impurities to Stimulate Stable Dehydrogenation Rates and Demonstrate that Deactivation and/or Lower Rates Prevail in the Absence of Clean Reactant Streams

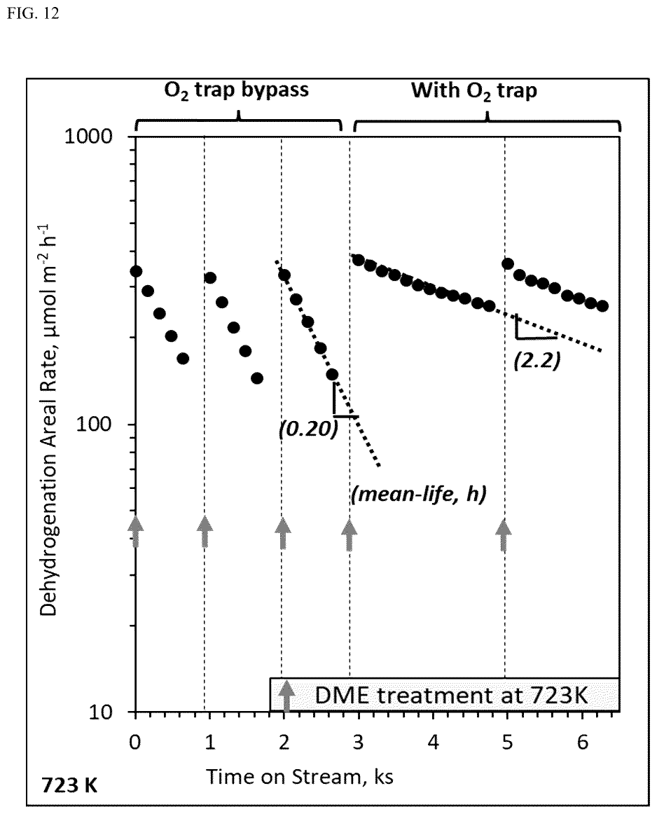

Rates of propane dehydrogenation (PDH) at a temperature of 723 K decreased with time on stream for DME-treated monoclinic ZrO₂ materials as prepared in Example 1a. The decrease in rate reflects the presence of trace amounts of titrant molecules (see Example 12 above) present in the feed stream as impurities. The first three panels in FIG. 12 show first-order catalyst deactivation arising from such impurities in the reactant stream, having a mean life of active sites of 0.2 h (defined as the reciprocal of the first order deactivation rate constant, ka). It has been found that the use of an oxygen trap (Agilent 5182-9401, 5 ppb specifications) to remove impurities from the reactant stream is advantageous. Including an oxygen trap for trapping such molecules when present in reactant gases before such gases can contact the catalyst decreases the slope of the deactivation curves and increases the active site mean life to 2.2 hours (right two panels of FIG. 12). The use of an oxygen trap extends the high rates afforded through thermochemical catalyst treatments by removing molecules from the feed stream that would render catalyst sites inactive for alkane dehydrogenation. Intervening DME treatments may restore rates, indicative of the role of impurities that DME can remove from the catalysts (and the trap from the stream) as the cause of the observed deactivation.

FIG. 12 illustrates propane dehydrogenation rates and the mean-life of active sites at 723 K for DME treated ZrO₂ of Example 1a with reactant stream bypassing or flowing through the trap. In FIG. 12, the use of an oxygen or water trap led to slower deactivation to an extent that is inversely proportional to the concentration levels of impurities in the inlet stream.

The impurity concentrations were also estimated from deactivation rate constants. The deactivation rate constants typically observed in these examples and the corresponding concentration of oxygen fed to the reactor will be described.

Equation 2 was used to calculate the rate for a first-order deactivation process:

$${r}_{dehyd} ( t ) = {r}_{dehyd , 0} {e}^{- {k}_{d} t} \qquad ( Equation 2 )$$

Where r is the rate of reaction, k_{d} is the deactivation rate constant, and t is time. Since the deactivation rate constant signifies the characteristic time for the decay of sites (ns), the concentration of the stoichiometric titrant is given by Equation 3:

$$[ titrant ] {F}_{0} = \frac{d {n}_{s}}{dt} = - \frac{( 1 - {e}^{- {k}_{d} t} ) {n}_{s , 0}}{dt} \qquad ( Equation 3 )$$

Where t is the time since deactivation initiated, dt is the interval for the decay and F₀ is the flow rate of gas across the catalyst bed. For the reaction conditions above, this provides an estimate for the concentration of titrant molecules shown in Table 2 below. The data in Table 2 illustrates the purity levels required in the reactant stream to maintain the very high rates made possible by the use of chemical treatment and by the use of high surface area catalysts with a high areal density of active sites uncovered by such chemical cleaning treatments. It is noted that the purity levels required in the present disclosure are sensitive and should be controlled to maintain a low oxygen concentration.

TABLE 2
Estimated Concentrations of Titrant Molecules

| | K_{d}, | Equivalent Inlet O₂ |
|---|---|---|
| Panel | ks⁻¹ | Concentration, ppm |
| 1 | 1.2 | 2.3 |
| 2 | 1.2 | 2.3 |
| 3 | 1.1 | 2.2 |
| 4 | 0.12 | 0.23 |
| 5 | 0.11 | 0.21 |

### Example 14: Activation-Deactivation of ZrO₂ (of Example 1a) and Y—ZrO₂ (of Example 2) Materials Based on Initial Treatment Conditions in the Presence of a Trap

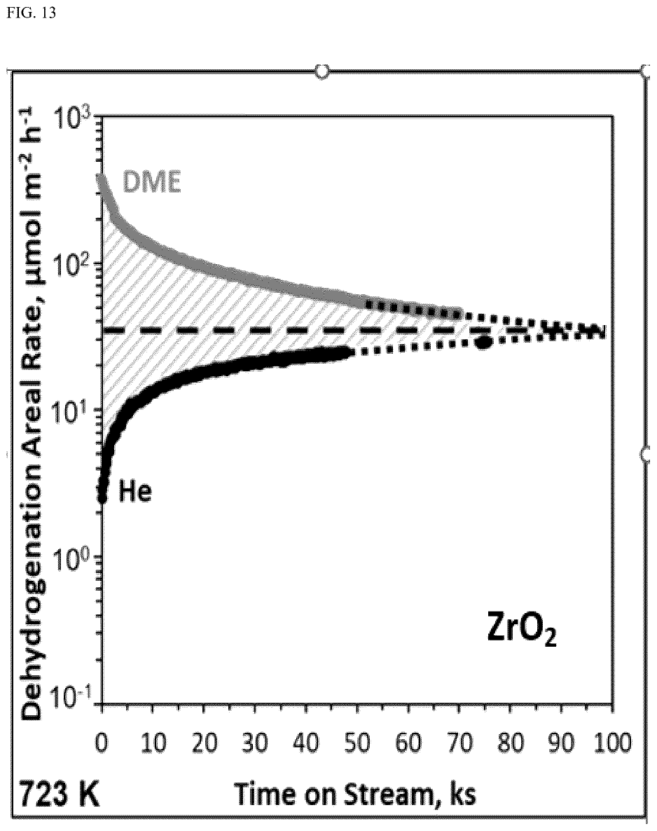

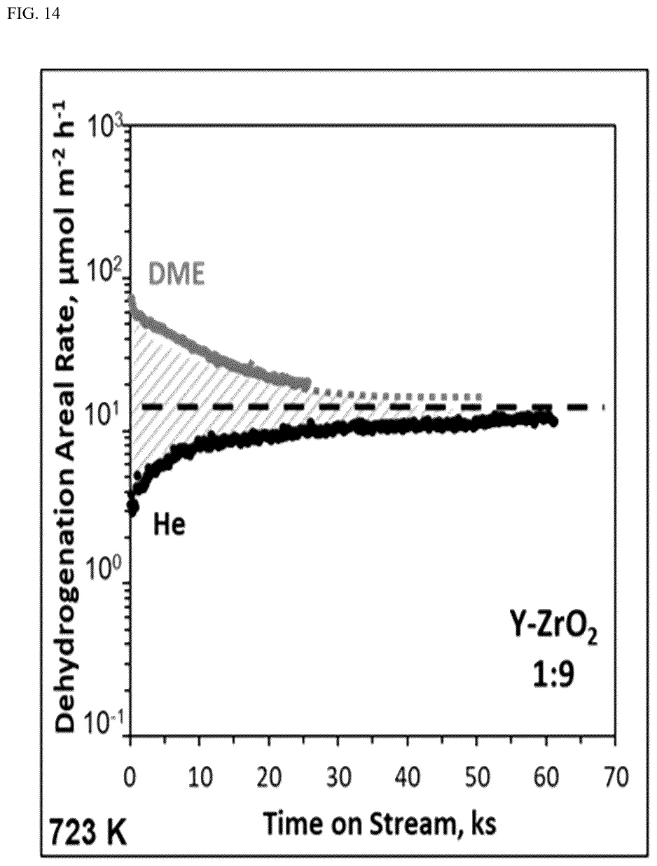

He treatments (at 723 K) led to lower propane dehydrogenation rates than on the same sample treated with DME (at 723 K) before reaction. It was found that rates after He treatment increased over time on stream as product propene molecules can act to partially uncover titrated sites from bound species that could not be removed by inert He at the same temperature as for propane dehydrogenation. Rates stabilized with time as a steady-state between site cleaning by reaction products and site titration by residual O₂ or H₂O in the feed stream (sub-ppm levels) is reached. In other words, the rates at which H₂O enters and titrates sites matches the rate at which any given propene concentration, defined by the propane conversion, is able to remove such titrants via chemical reactions. In contrast, materials initially treated with DME (at 723 K) exhibit dehydrogenation rates which decrease with time on stream as residual O₂ or H₂O in the feed stream (sub-ppm levels) titrate sites until the same steady-state between site cleaning (activation) and titration (deactivation) is also reached. The steady-state rates after each treatment and for each material are equivalent for the same experimental conditions but the approach to such a steady-state occurs from higher or lower rates, depending on the initial extent of dehydroxylation caused by the initial treatment in He or DME. These steady-state rates are lower for higher inlet H₂O levels than for lower ones, illustrative of the need for controlled purity levels in reactant streams. This demonstrates the requirement for the strict removal of titrants or their precursors to achieve higher steady-state reaction rates and also the benefits of a DME treatment which allows a larger number of catalytic turnovers (as seen by the integrated area between the two curves) before both samples reach steady-state rates. These is seen in FIGS. 13 and 14, which shows that this is the case for both YZr and Zr oxides.

### Example 15: Effect of Oxygen Trap on Catalyst Self-Activation and Deactivation at 723 K and 873 K

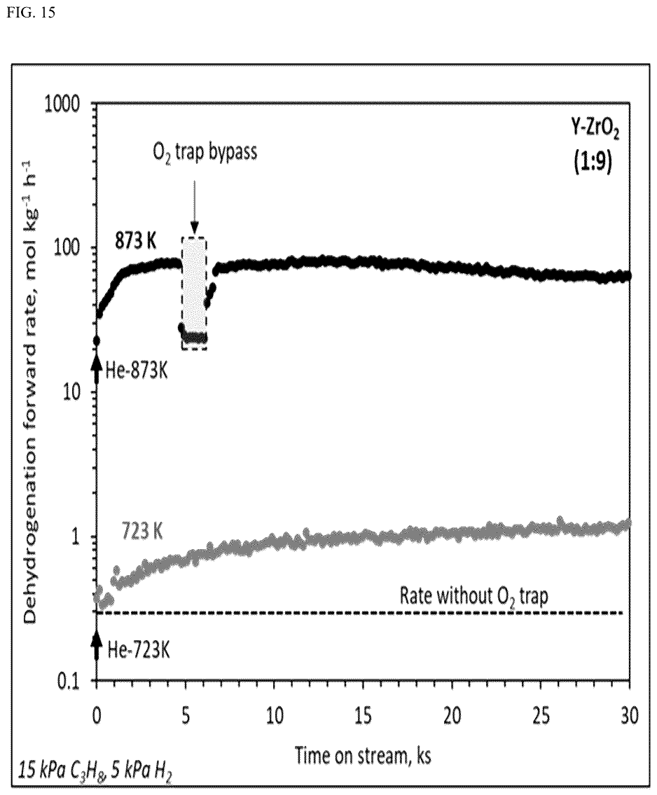

The presence of an oxygen trap enables self-activation at both 723 K and 873 K. Bypassing the oxygen trap decreases reaction rates as the concentration of titrants in the feed stream increases to suppress dehydrogenation reactions. Upon cleaning the reactant stream, rates increased as reaction products serve as cleaning agents. This reflects a new steady-state extent of dehydroxylation imposed by higher impurity levels when the inlet stream bypassed the trap and can be seen in FIG. 15. In FIG. 15, the oxygen trap was bypassed at indicated locations which showed lower dehydrogenation rates. FIG. 15 further demonstrates that the removal of impurities achieves higher steady-state rates as a result of a lower rate of titrant/impurity introduction. This balance through the removal of impurities by propene during reaction at temperatures where propene becomes competent, allowing propene to become an indigenous chemical dehydroxylation reagent that is formed during propane dehydrogenation.

### Example 16: Effect of Oxygen Trap on Propane Dehydrogenation Rate as the Reaction Temperature Increased from 723 to 873 K

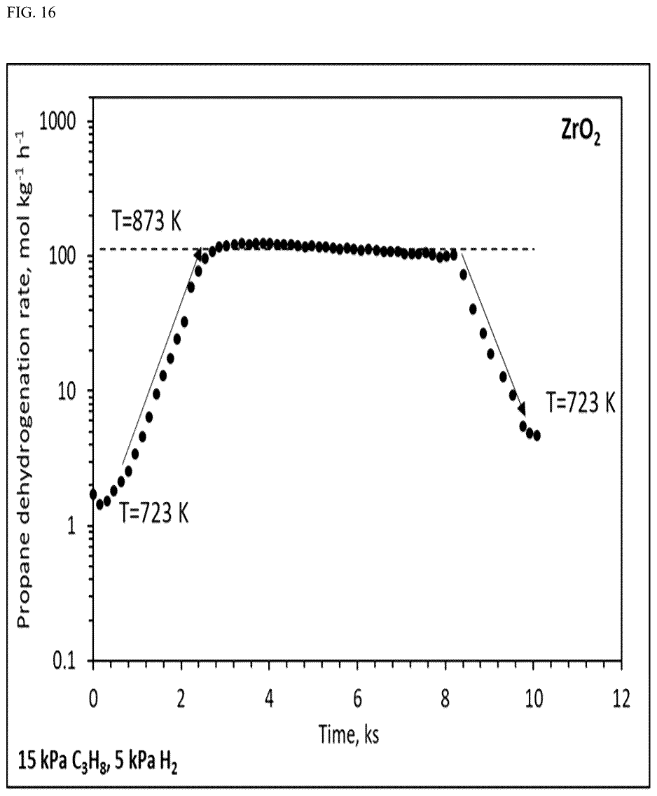

In FIG. 16, propane dehydrogenation rates on ZrO₂ were measured upon increasing the temperature from 723 K to 873 K during propane dehydrogenation with the reactant stream passing through the oxygen trap at the following reaction conditions: 15 kPa C₃H₈, 5 kPa H₂. With increasing dehydrogenation reaction temperatures, it has been found that the reaction rate increases as is shown in FIG. 16. The present study combines both the temperature effect on reaction rates and the catalyst self-activation by the reaction product, which becomes more effective as temperature increased. Decreasing the temperature back to 723 K resulted in a higher dehydrogenation rate than measured at 723 K before experiments at 873 K, indicative of the propene reaction products acting as a competent dehydroxylation agent at these higher temperatures. The presence of the oxygen trap avoids catalyst deactivation and enables the catalyst to achieve higher steady-state dehydrogenation rates.

### Example 17: Representative Propane Dehydrogenation Rates with and without an Oxygen Trap According to an Embodiment of the Present Disclosure at Different Temperatures and for Different Catalysts

Table 3 illustrates the comparison of propane dehydrogenation rates.

TABLE 3
Comparison of Propane Dehydrogenation rates

| | | Pretreatment | Reaction | No O₂ trap | With O₂ trap |
|---|---|---|---|---|---|
| | | and reaction | temperature, | Dehydrogenation | Dehydrogenation |
| Sample | Catalyst | conditions | K | rate, mol kg⁻¹ h⁻¹ | rate, mol kg⁻¹ h⁻¹ |
| E1 | ZrO₂ | He 723 K | 723 | 0.33 | 3.9 |
| E1 | ZrO₂ | DME 723 K | 723 | 31 | 36 |
| E1 | ZrO₂ | He 723 K | 873 | — | 122 |
| E2 | Y—ZrO₂ | He 723 K | 723 | 0.37 | 1.4 |
| | (1:9) | | | | |
| E2 | Y—ZrO₂ | DME 723 K | 723 | | 8.2 |
| | (1:9) | | | | |
| E2 | Y—ZrO₂ | He 873 K | 873 | 23 | 80 |
| E3 | CoO/SiO₂ | O₂ 873 K | 873 | 9.2 | 16.3 |
| E4 | Al₂O₃ | He 873 K | 873 | | 28 |
| S1 | ZrO₂ | — | 863 | 2.5 | |
| S1 | ZrO₂ | — | 893 | 4.3 | |
| S2 | Y—ZrO₂ | — | 893 | 35% conversion | |
| | | | | LHSV = 3 | |
| S4 | Commercial | — | 893 | 40% conversion | |
| | | | | LHSV = 3 | |
| S5 | ZrO₂ | Reduction | 823 | 11.25 | |
| | | in H₂ | | | |
| S5 | ZrO₂ | Reduction | 823 | 33.75 | |
| | | in CO | | | |

Sample E1 is a sample as prepared according to Example 1a;
Sample E2 is a sample as prepared according to Example 2;
Sample E3 is a sample as prepared according to Example 3;
Sample E4 is a commercially available Al₂O₃ material: Catalox SBa-200 from Sasol S1, S2 and S4 are samples as prepared according to Pan et al. (US 2009/0325784);
S5 are samples as prepared according to Zhang et al. “Control of Coordinatively Unsaturated Zr Sites in ZrO₂ for Efficient C—H Bond Activation

### Example 18: Prereduction Using Reducible Oxide

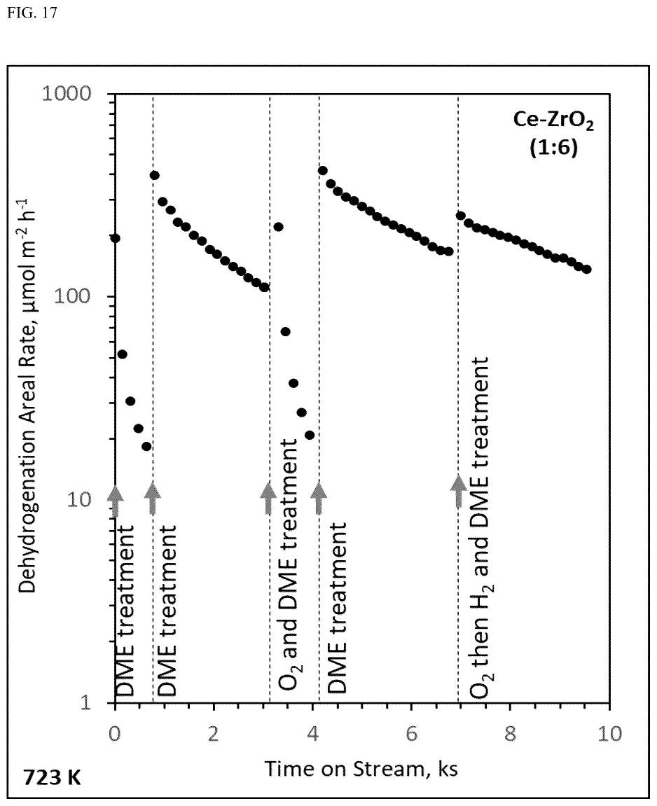

For reducible oxides, such as Ce—ZrO₂ (Ce—Zr ratio 1:6), reduction pretreatments (e.g. with H₂, 723 K) are required before DME treatments which cleanse sites, otherwise reduction of the reducible component of the catalyst under dehydrogenation reaction conditions results in the formation of H₂O during alkane dehydrogenation, which titrates the active LAB sites and deactivates the catalyst. The results of this study is presented in FIG. 17. After the first DME treatment, the catalyst rapidly deactivated because CeO₂ reduction during dehydrogenation, resulting in water formation and active site titration. After cleaning titrated sites by the second DME treatment, deactivation rate decreased because Ce is already reduced and there is no more extra water formation. After oxidizing the catalyst followed by DME treatment the same effect was observed (panel 3 and 4 compared to the panels 1 and 2). However, if after oxidation, reductive treatment is performed before DME treatment (CeO₂ is reduced and the formed water removed by the DME treatment), deactivation rate is smaller (panel 5), because all CeO₂ is already reduced during the reductive treatment and there is no water formation during dehydrogenation.

### Example 19: Enhanced Reaction Rates Through Chemical Cleaning were not Caused by Reduction of Oxide Catalysts

This test was conducted in the absence of an oxygen trap during purging periods. It was found that the rates, which was enhanced by DME treatments, were not enhanced by He or H₂ treatments. This test was conducted to compare the chemical treatments presented so far in the literature, reductive treatment of surfaces versus a dehydroxylating method.

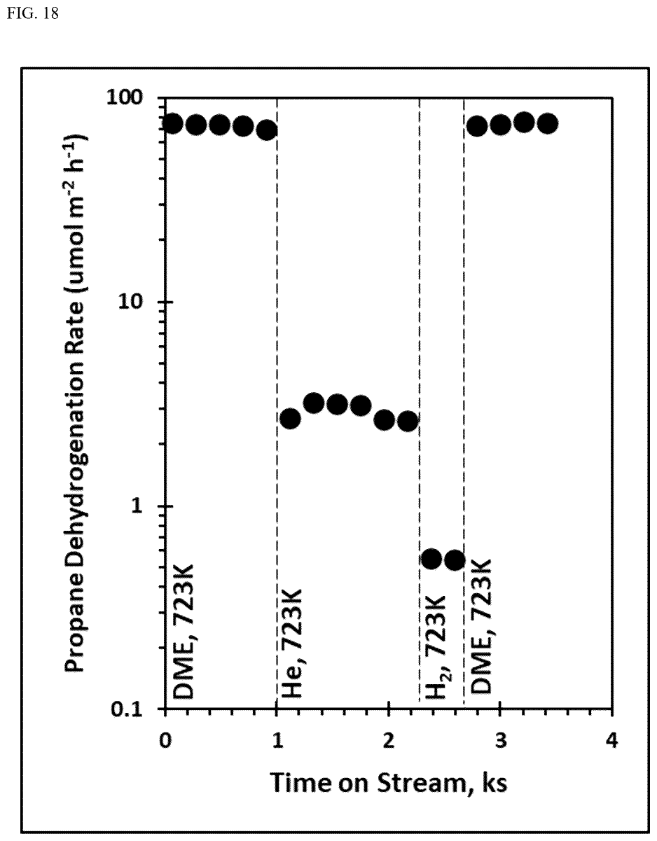

As can be seen in FIG. 18, reductive treatment of surfaces is not beneficial for the cleansing of metal oxides. In FIG. 18, propane dehydrogenation rates after DME, He and H₂ treatments are shown at the following conditions: 13.7 kPa propane, 723 K. A sample according to Example 1a of the present application was treated with H₂ along with S5 as described in Table 3 above. After treatment, the activity of the material was less than after the treatment with DME or He. Thus, the inventors believe this indicates that the activity of the materials was not because of the reduced metal centers, but because of the bare Lewis acid-base pairs exposed on the surface. The oxygen trap in the catalyst keeps the surface clean.

The present inventors estimated the activity of comparative examples, based on literature thermodynamic data, which confirm the unlikely reduction of the metal cations of the metal oxide materials.

The formation of an oxygen vacancy by reactions with hydrogen to form water is unlikely under the conditions of the present dehydrogenation reactions. The required reduction potential, depicted as a H₂ to H₂O ratio, for these oxides was computed by extracting DFT-derived oxygen vacancy formation energy from literature. The remaining energetics required to form water from molecular H₂ and diatomic O was computed from tabulated thermochemical databases. Combining these values enable the prediction of free energies for the reaction at 873 K and subsequently equilibrium constants, which are used to derive the H₂ to H₂O ratios necessary for O removal. The calculations were performed using Equations 4 to 7 presented below.

$${MO}_{X} + {H}_{2} \to {MO}_{X - 1} + {H}_{2} O \qquad ( Equation 4 )$$

$$Δ {G}_{v} = Δ {G}_{f , {MO}_{X - 1}} + Δ {G}_{f , {H}_{2} O} - Δ {G}_{f , {MO}_{X}} - Δ {G}_{f , {H}_{2}} \qquad ( Equation 5 )$$

$$Δ {G}_{v} = ( Δ {G}_{f , {MO}_{X - 1}} - Δ {G}_{f , M {O}_{X}} ) + Δ {G}_{f , {H}_{2} O} - Δ {G}_{f , O} - Δ {G}_{f , {H}_{2}} \qquad ( Equation 6 )$$

Where National Institute of Standards and Technology (NIST) and Stull, Westrum and Sinke “The chemical thermodynamics of organic compounds” (Stull) give: Δ_{G}_{_{f}}_{,H}_{₂}_{O}−ΔG_{f,O}−ΔG_{f,H}_{₂}(873 K)=−156.3 kJ mol⁻¹

$$K = \frac{[ {MO}_{X - 1} ] [ {H}_{2} O ]}{[ {MO}_{X} ] [ {H}_{2} ]} = {e}^{\frac{- Δ {G}_{r e d}}{R T}} \qquad ( Equation 7 )$$

The resulting H₂ to H₂O ratios necessary to stimulate oxygen removal from the lattice to form active sites are shown in Table 4. Thus, it can be confirmed that oxygen removal from the lattice to form a vacancy is unlikely under the dehydrogenation reaction or pretreatment conditions. As can be seen in Table 4, the ratios are very high are would not be attainable using any available methods of water removal protocols. In fact, the ratio requires, for at least the first four samples in Table 2, fewer than one molecule of water per mole of H₂¬ (order of parts per octillion).

TABLE 4
Oxygen Vacancy generation energies and
H₂/H₂O ratios required for vacancy generation

| | E_{f} neutral | ΔG_{v, 873 K} | H₂/H₂O |
|---|---|---|---|
| System | (eV) | kJ mol⁻¹ | Ratio |
| ZrO₂ (bulk)⁵ | 6.16 | 438 | 1 · 10²⁶ |
| m- ZrO₂ (101)⁵ | 6.03 | 425 | 3 · 10²⁵ |
| MgO⁵ | 6.19 | 440 | 2 · 10²⁶ |
| Y₂O₃⁶ | 6.92 | 511 | 4 · 10³⁰ |
| CoO monomer | Not Available | | |
| TiO₂⁵ | 4.53 | 281 | 6 · 10¹⁶ |
| CeO₂⁸ | 2.44 | 79 | 5 · 10⁴ |

⁵refers to Puigdollers et al. “Increasing Oxide Reducibility: The Role of Metal/Oxide Interfaces in the Formation of Oxygen Vacancies.” Acs Catal 2017, 7, 6493-6513
⁶refers to Zheng et al. “Native Point Defects in Yttria and Relevance to Its Use as a High-Dielectric-Constant Gate Oxide Material: First Principles Study” Physical Review B 2006, 73
⁸Lawler et al. “CeO₂ (111) Surface with Oxygen Vacancy for Radical Scavenging: A Density Functional Theory Apprach” J. Phys. Chem C 2020, 124, 20950-20959
E_{f} is the DFT-derived electronic energies required to remove each O atom from the stoichiometric oxide in its highest oxidation state.

### Example 20: Hydrogenation of Unsaturated Hydrocarbons

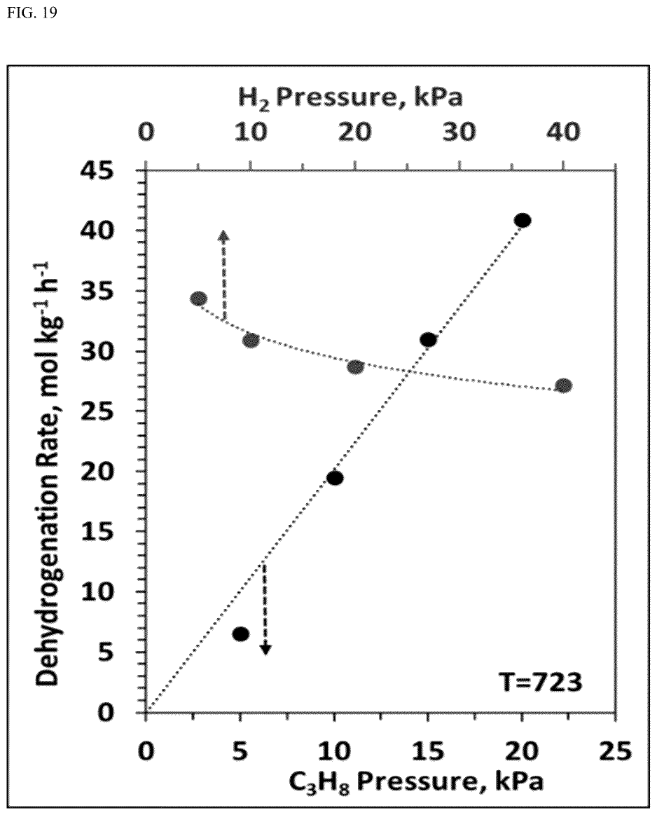

As described herein, the present application is directed to avoiding contact with impurities to prevent Lewis acid-base pair active sites from being deactivated through titration of the most competent active centers for dehydrogenation processes. It has been surprisingly found that these considerations may also be applied for the reverse reactions. This is believed to be possible because both processes involve the same rate-limiting step and transition state, but traverse the reaction coordinate in opposite directions. (Gounder et al. “Catalytic Hydrogenation of Alkenes on Acidic Zeolites: Mechanistic Connections to Monomolecular Alkane Dehydrogenation Reactions” J Catalyst 2011, 277, 36-45.) This extension applies to systems in which:
Metal oxide Lewis acid-base pairs which dehydrogenate alkanes are also capable of hydrogenating unsaturated hydrocarbons (e.g. alkenes and alkynes, shown for propene in FIGS. 19-21) with thermodynamic connections between the two reactions due to meeting strict criterion of the hydrogenation and dehydrogenation chemistry on the Lewis acid-base pairs.
For each given hydrogenated-dehydrogenated pair (e.g. alkane-alkene pair), the rates of dehydrogenation to hydrogenation (and of their pressure dependence) reflect the gas phase thermodynamics of their interconversion reaction. Thus, enabling prediction of the rates for the reverse reaction
Thermochemical treatments (e.g. DME, 723 K) and stream cleaning protocols that are competent at enhancing the reaction rates for dehydrogenation reactions also enhance hydrogenation reaction rates, though may be less necessary if the reactant is also a chemical activator (e.g. alkene)

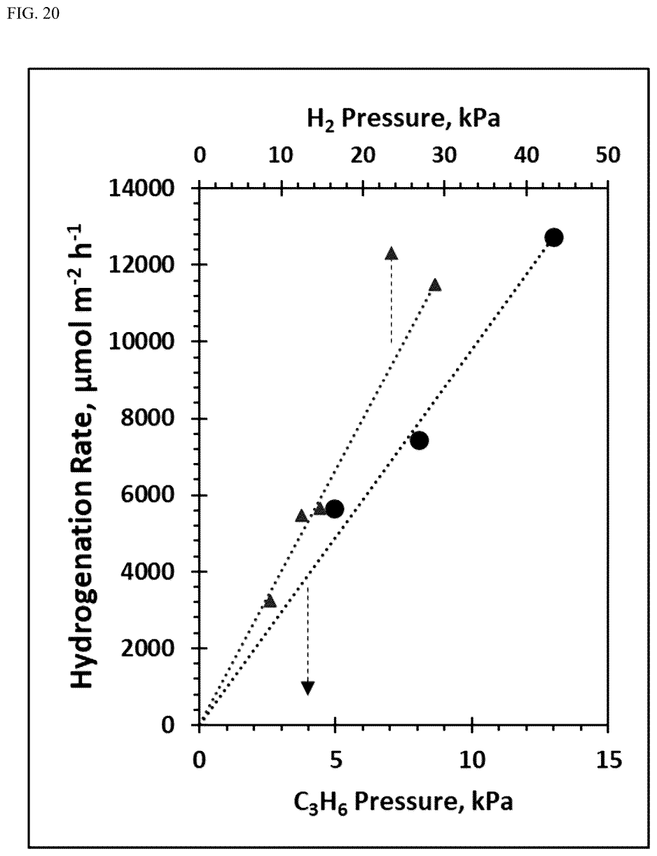

For example, a representative reaction is that of propene hydrogenation on monoclinic ZrO₂ catalysts. Rates are first order with respect to both propene and hydrogen pressures (FIGS. 19 and 20), consistent with a reaction rate equation:

$${r}_{hyd} = {k}_{hyd} [ {C}_{3} {H}_{6} ] [ {H}_{2} ] \qquad ( Equation 8 )$$

Using the knowledge about reversibility described above, which require essentially bares sites and mechanisms that proceed through identical kinetically relevant transition state, rates of hydrogenation and dehydrogenation can be linked to the reverse reaction through the gas phase reaction equilibrium constant according to Equation 9:

$${K}_{dehyd} = \frac{{r}_{dehyd}}{{r}_{hyd}} = \frac{{k}_{dehyd} [ {C}_{3} {H}_{8} ]}{{k}_{hyd} [ {C}_{3} {H}_{6} ] [ {H}_{2} ]} \qquad ( Equation 9 )$$

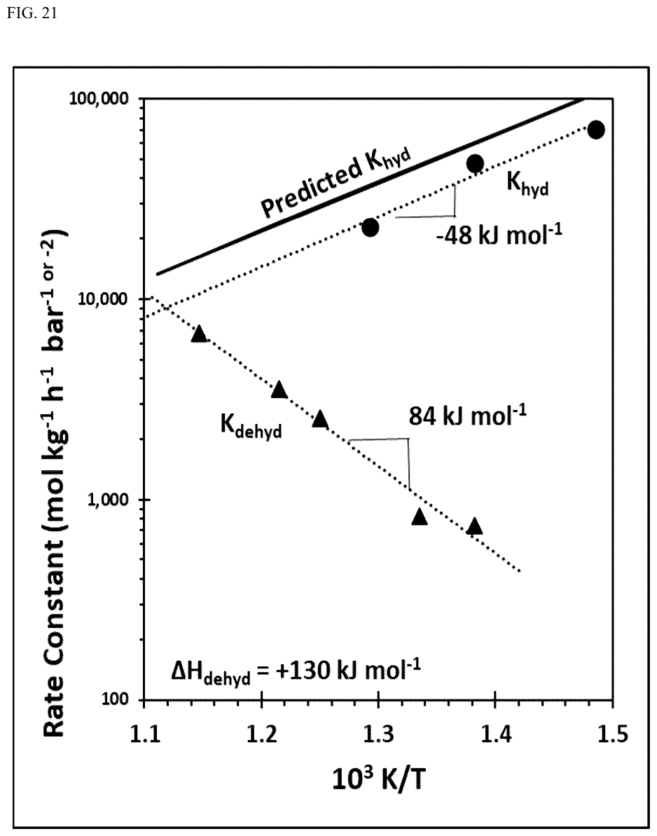

The Lewis acid-base pairs shown as examples here meet this strict requirement and thus, rates of propene hydrogenation can be predicted from propane dehydrogenation rates (FIG. 21) and are reasonably consistent with those measured (circles) for ZrO₂ catalysts. The difference between the measured barriers for each reaction (84 and −48 kJ mol⁻¹) is consistent with the heat of reaction (130 kJ mol⁻¹).

1. A method of treating a reactant stream, comprising:
treating the reactant stream with a MOₓ catalyst and a trap, wherein the trap removes an impurity from the reactant stream, the impurity comprising oxygen (O₂), water (H₂O), hydrogen sulfide (H₂S), carbon dioxide (CO₂), carbon monoxide (CO), nitrogen (N₂), a sulfur containing compound, methanol, a ketone, an inorganic nitrogen compound, an organic nitrogen compound, an oxygenate, or a combination thereof;
wherein the MOₓ catalyst has a surface with a M-O site of the Lewis type and of balanced acid-base strength, and
wherein the MOₓ catalyst has a BET surface area of at least about 75 m²/gram.

2. The method of claim 1, wherein the impurity is removed from a reactant stream before contacting the catalyst composition during a hydrogenation or dehydrogenation process.

3. The method of claim 1, wherein a surface of the MOₓ catalyst stabilizes anionic and/or cationic moieties that form at transition states for heterolytic processes that form and cleave C—H bonds.

4. The method of claim 1, wherein a metal (M) of the MOₓ catalyst does not undergo reduction to a lower oxidation state in a reductive environment typical of hydrogenation-dehydrogenation catalysis.

5. The method of claim 1, wherein the method operates at an impurity content of less than about 2 ppm.

6. (canceled)

7. The method of claim 1, wherein the reactant stream has an oxygen content of at most 5 ppm.

8. The method of claim 1, wherein the surface area of the MOₓ catalyst is proportional to number of actives of the MOₓ catalyst.

9. The method of claim 1, wherein the MOₓ catalyst comprises a crystalline active component, wherein when the surface area of the crystalline active component decreases, then a density of active sites of the MOₓ catalyst decreases.

10. The method of claim 1, wherein the MOₓ catalyst is substantially free of hydroxyl groups.

11.-13. (canceled)

14. The method of claim 1, wherein the treating is performed at a temperature of about 500-900 K.

15. (canceled)

16. The method of claim 1, wherein the MOₓ catalyst comprises a metal oxide having metal centers in cationic form.

17. The method of claim 1, wherein the MOₓ catalyst comprises a metal oxide including cations that are not reducible to a zero-valent state.

18. The method of claim 1, wherein the MOₓ catalyst comprises a metal selected from the group consisting of zirconium (Zr), cobalt (Co), gallium (Ga), zinc (Zn), cerium (Ce), yttrium (Y), and titanium (Ti).

19. The method of claim 1, wherein the MOₓ catalyst comprises one or more of Mg, Ca, Sr, Ba and La on a zirconia support.

20. The method of claim 1, wherein the MOₓ catalyst comprises ZrO₂-silica, Zr—Al, Zr—Ti, or a combination thereof.

21. The method of claim 1, wherein the MOₓ catalyst comprises ZrO₂, t-ZrO₂, m-ZrO₂, Y-stabilized ZrO₂, or Y₂O₃.

22.-30. (canceled)

31. A method of catalyzing a reaction using a catalyst composition comprising a MOₓ catalyst and a trap, comprising activating and/or reactivating the catalyst composition using the method of claim 1, wherein the reaction is selected from the group consisting of alkane dehydrogenation, alkene hydrogenation, olefin-paraffin alkylation, from CO/H₂ mixtures without O-rejection as H₂O or CO₂, C—C bond formation via alkene oligomerization or metathesis, dehydrocyclization (alkanes/alkenes to arenes), dehydrocyclodimerization (alkanes/alkenes to arenes with a larger number of C-atoms), transfer hydrogenation, hydroformylation/carbonylation, aromatization, dearomatization, reforming, isomerization, and bifunctional reactions in which one of the aforementioned functions can be combined with a Brønsted acid function.

32.-39. (canceled)

40. A catalyst composition comprising:
a MOₓ catalyst having a surface with an M-O site of the Lewis type and of balanced acid-base strength and a BET surface area of at least about 50 m²/gram,
wherein the catalyst composition is free of at least one of chromium or a precious metal.

41. The catalyst composition of claim 40, wherein the MOₓ catalyst comprises a metal oxide having metal centers in cationic form.

42. The catalyst composition of claim 40, wherein the MOₓ catalyst comprises a metal oxide that is not reducible to zero-valent state.

43.-51. (canceled)
